(19) United States
(12) Patent Application Publication
(10) Pub. No.: US 2026/0348959 A1
(43) Pub. Date: Nov. 5, 2026

(54) METHOD FOR THE DETECTION OF ANTI-AAV PARTICLE ANTIBODIES

(71) Applicant: Hoffmann-La Roche Inc., Little Falls, NJ (US)

(72) Inventors: Kay-Gunnar STUBENRAUCH, Penzberg (DE); Uwe WESSELS, Penzberg (DE)

(73) Assignee: Hoffmann-La Roche Inc., Little Falls, NJ (US)

(21) Appl. No.: 19/291,793

(22) Filed: Aug. 6, 2025

Related U.S. Application Data
(63) Continuation of application No. PCT/EP2024/052686, filed on Feb. 5, 2024.

(30) Foreign Application Priority Data
Feb. 7, 2023 (EP) 23155243.1

(51) Int. Cl. G01N 33/569

(52) U.S. Cl. CPC G01N 33/56983; G01N 2333/015; G01N 2469/20

(57) ABSTRACT

Herein is reported a method for determining an anti-AAV particle antibody in a mammalian sample comprising the steps of diluting an aliquot of the sample, preferably to a final serum concentration between 5% (v/v) and 0.5% (v/v), adding AAV particles (AAVp) specifically bound by the anti-AAV particle antibody (AAV-ADA) to the diluted sample to generate a spiked sample, pre-incubating the spiked sample to allow the formation of a AAV-ADA-AAVp complex, and detecting the AAV-ADA-AAVp complex, thereby determining the anti-AAV antibody in the mammalian sample.

### CROSS REFERENCE TO RELATED APPLICATIONS

This application is a continuation of PCT Application No. PCT/EP2024/052686, filed Feb. 5, 2024, which claims priority to EP Application Serial No. 23155243.1, filed Feb. 7, 2023, each of which is incorporated herein by reference in its entirety.

### BACKGROUND OF THE INVENTION

The current invention is in the field of immunoassays, especially in the field of anti-drug antibody (ADA) assessment. In many cases, ADA assessment is challenging, especially in studies that involve the administration of adeno-associated virus-based gene therapies. In such cases, ADA assays with low reagent demand are desired due to the limited availability of the therapeutic AAV as assay reagent. The current invention is based, at least in part, on the finding that complexes comprising the AAV-ADA can be generated for detection by pre-incubating a sample, in which anti-AAV particle antibodies (AAV-ADAs) have to be determined, with the respective therapeutic AAV particle. With such an approach the demand of the therapeutic AAV particle compared to conventional ADA assays requiring the labeling of the therapeutic AAV particle are dramatically reduced.

Immunogenicity assessment of therapeutic drug candidates is an important part of the drug development process. In cases of an immune response, appropriate interpretation of immunogenicity data is required to enable a correlation with clinical outcomes. Bioanalytical methods used for immunogenicity testing provide the required information by detecting and characterizing anti-drug antibodies (ADAs).

Like all protein therapeutics, recombinant AAV particles have to be analyzed prior to their use. However, in contrast to other biotherapeutics, adeno-associated viral therapies are hampered by a pre-existing humoral immunity against AAVs (anti-AAV particle antibodies, AAV-ADAs) due to the ubiquitous nature of AAVs and the concurrent wide distribution of pre-therapeutic AAV infection in the human population.

For normal biotherapeutics the “gold standard” assay format for detecting ADAs in human or monkey samples is the bridging assay. Therein ADAs are complexed with labeled drug conjugates to form signal-giving complexes. Due to the assay setup differently labeled drug molecules or a surface coated with the drug are needed. This requires substantial amounts of the drug.

Gimpel, A. L., et al. (Mol. Ther. Meth. Clin. Dev. 20 (2021) 740-754) summarized the drawbacks of current AAV analytical methods used for the analysis of recombinantly produced AAV particles:
Commonly, laboratory-scale production yields only a few milliliters (mL) of purified rAAV particles—equivalent to 1E12-1E14 vector genomes (vg)—such that the required sample volumes for analysis can cause significant loss of product. At the same time, virus concentration in samples covers many orders of magnitude, from 1E10 to 1E11 vg/mL at raw culture harvest to 1E13 to 1E14 vg/mL in the purified product and the sample matrix varies significantly, with the mass concentration of residual byproducts, host cell proteins, and DNA exceeding that of rAAV by 10- to 100-fold in initial downstream steps.

Thus, the analytical characterization of recombinantly produced AAV particles as such and the analytics accompanying development require a substantial amount of the product and thereby reducing its availability for characterization and testing.

Gorovits et al. (The AAPS Journal 23 (2021) 108) reviewed important considerations related to anti-AAV total antibody detecting assays. In FIG. 1 of Gorovits, the different applied total anti-AAV antibody assay formats are summarized. In the generally used antigen-capture and bridging formats, the viral capsid is immobilized either directly or via a specific binding pair as capture reagent on a solid surface. This setup is used in ELISA and ECLIA format with different detection reagents, such as a HRP-conjugated anti-species Ig antibody as well as HRP-conjugated protein A or G (antigen capture format) or labeled viral capsid (bridging format).

Thus, there is a need for AAV-ADA assay with reduced AAV material demands.

### SUMMARY OF THE INVENTION

Herein reported are novel methods for the determination of anti-AAV particle antibodies (AAV-ADAs) in a sample. The methods according to the current invention require amongst other things reduced AAV material amounts compared to established assays.

The current invention is based, at least in part, on the finding that by pre-incubating a sample, in which AAV-ADAs have to be determined, with the respective therapeutic AAV particle complexes comprising the AAV-ADAs can be generated for detection. Thereby the demand, i.e. the required amount, of the therapeutic AAV particle as assay reagent compared to state of the art AAV-ADA assays wherein labeled therapeutic AAV particle is employed can be dramatically reduced.

The current invention encompasses at least the following embodiments:
Embodiment 1: A method for measuring an anti-AAV particle antibody in a mammalian serum sample comprising the following steps in the following order:
   a) adding AAV particles (AAVp) specifically bound by the anti-AAV particle antibody (AAV-ADA) to the sample to generate a spiked sample,
   b) pre-incubating the spiked sample to allow the formation of a rAAV-ADA-AAVp complex,
   and
   c) detecting the AAV-ADA-AAVp complex, thereby measuring the anti-AAV antibody in the mammalian serum sample.
Embodiment 2: The method according to embodiment 1, wherein the method further comprises a step bc) after step b) and before step c) of
   bc) adding a protein or polypeptide specifically binding to the AAVp that is conjugated to a first member of a binding pair (captAb-BI) to the pre-incubated spiked sample and incubating the mixture to allow the formation of a ternary AAV-ADA-AAVp-captAb-BI complex,
   and step c) is
   c) detecting the ternary AAV-ADA-AAVp-captAb-BI complex.
Embodiment 3: The method according to any one of embodiments 1 to 2, wherein the method further comprises a step bbc) after step b) or after step bc) and before step c) of
   bbc) capturing the ternary complex formed in step bc) on a solid surface.
Embodiment 4: A method for measuring an anti-AAV particle antibody in a mammalian serum sample comprising the following steps in the following order:
   a) diluting at least an aliquot of the sample,
   b) adding AAV particles (AAVp) specifically bound by the anti-AAV particle antibody (AAV-ADA) to the diluted sample to generate a spiked sample,
   c) pre-incubating the spiked sample to allow the formation of an AAV-ADA-AAVp complex,
   and
   d) detecting the AAV-ADA-AAVp complex,
   thereby measuring the anti-AAV antibody in the mammalian sample.
Embodiment 5: The method according to embodiment 4, wherein the method further comprises a step cd) after step c) and before step d) of
   cd) adding a protein or polypeptide specifically binding to the AAVp that is conjugated to a first member of a binding pair (captAb-BI) to the pre-incubated spiked sample and incubating the mixture to allow the formation of a ternary AAV-ADA-AAVp-captAb-BI complex,
   and step d) is
   d) detecting the ternary AAV-ADA-AAVp-captAb-BI complex.
Embodiment 6: The method according to any one of embodiments 4 to 5, wherein the method further comprises a step ccd) after step c) or after step cd) and before step d) of
   ccd) capturing the complex formed in step c) on a solid surface.
Embodiment 7: The method according to any one of embodiments 4 to 6, wherein the diluting is to a final mammalian serum concentration of less than 50% (v/v) and 0.5% (v/v) or more.
Embodiment 8: The method according to any one of embodiments 4 to 7, wherein the diluting is to a final mammalian serum concentration of less than 20% (v/v) and 0.5% (v/v) or more.
Embodiment 9: The method according to any one of embodiments 4 to 8, wherein the diluting is to a final mammalian serum concentration of less than 5% (v/v) and 0.5% (v/v) or more.
Embodiment 10: The method according to any one of embodiments 1 to 9, wherein the measuring is determining.
Embodiment 11: The method according to any one of embodiments 1 to 9, wherein the measuring is quantifying.
Embodiment 12: The method according to any one of embodiments 1 to 11, wherein the pre-incubating is for 5 to 90 minutes.
Embodiment 13: The method according to any one of embodiments 1 to 12, wherein the pre-incubating is for 15 to 60 minutes.
Embodiment 14: The method according to any one of embodiments 4 to 13, wherein the pre-incubating is for 5 to 25 minutes and the final mammalian serum concentration is about 2% (v/v).
Embodiment 15: The method according to any one of embodiments 4 to 13, wherein the pre-incubating is for 26 to 90 minutes and the final mammalian serum concentration is about 1% (v/v).
Embodiment 16: The method according to any one of embodiments 1 to 15, wherein the final concentration of the AAVp in the spiked samples is in the range from 0.5 E9 to 1 E10 viral particles per mL spiked sample volume.
Embodiment 17: The method according to any one of embodiments 1 to 16, wherein the final concentration of the AAVp in the spiked samples is in the range from 1 E9 to 7 E9 viral particles per mL spiked sample volume.
Embodiment 18: The method according to any one of embodiments 1 to 17, wherein the final concentration of the AAVp in the spiked samples is in the range from 1.25 E9 to 5 E9 viral particles per mL spiked sample volume.
Embodiment 19: The method according to any one of embodiments 1 to 18, wherein the final concentration of the AAVp in the spiked samples is in the range from 1.5 E9 to 3.5 E9 viral particles per mL spiked sample volume.
Embodiment 20: The method according to any one of embodiments 1 to 19, wherein the sample volume or the volume of the aliquot of the sample is 100 μL or less.
Embodiment 21: The method according to any one of embodiments 1 to 20, wherein the sample volume or the volume of the aliquot of the sample is 50 μL or less.
Embodiment 22: The method according to any one of embodiments 1 to 21, wherein the sample volume or the volume of the aliquot of the sample is 10 μL or less.
Embodiment 23: The method according to any one of embodiments 1 to 22, wherein the sample volume or the volume of the aliquot of the sample is 5 μL or less.
Embodiment 24: The method according to any one of embodiments 1 to 23, wherein the sample volume or the volume of the aliquot of the sample is 1.5 μL or less.
Embodiment 25: The method according to any one of embodiments 1 to 24, wherein the sample volume or the volume of the aliquot of the sample is about 1 μL.
Embodiment 26: The method according to any one of embodiments 1 to 25, wherein the AAV-ADA specifically binds to an AAVp of the serotype 2 or 8 or 9 or rh74 or a variant thereof.
Embodiment 27: The method according to any one of embodiment 1 to 26, wherein the AAV-ADA is an anti-recombinant AAV particle antibody (rAAV-ADA).
Embodiment 28: The method according to any one of embodiment 1 to 27, wherein the AAV-ADA is an anti-therapeutic AAV particle antibody (tAAV-ADA).
Embodiment 29: The method according to any one of embodiment 1 to 28, wherein the AAV-ADA is an anti-recombinant therapeutic AAV particle antibody (rtAAV-ADA).
Embodiment 30: The method according to any one of embodiments 1 to 29, wherein the AAVp is conjugated to a first member of a binding pair.
Embodiment 31: The method according to any one of embodiments 2 to 3 and 5 to 30, wherein the captAb-BI is selected from the group consisting of anti-AAVp antibodies, AAVp-binding anticalins and AAVp-binding fragments thereof.
Embodiment 32: The method according to claim any one of embodiments 2 to 3 and 5 to 31, wherein the captAb-BI an anti-AAVp antibody or AAVp-binding fragment thereof.
Embodiment 33: The method according to any one of embodiments 2 to 3 and 5 to 32, wherein the captAb-BI is added to a final concentration of 0.1 to 0.5 μg/mL.
Embodiment 34: The method according to any one of embodiments 2 to 3 and 5 to 33, wherein the captAb-BI is added to a final concentration of 0.15 to 0.35 μg/mL.
Embodiment 35: The method according to any one of embodiments 2 to 3 and 5 to 34, wherein the captAb-BI is added to a final concentration of about 0.25 μg/mL.
Embodiment 36: The method according to any one of embodiments 2 to 3 and 5 to 35, wherein after the addition of the captAb-BI in step bc) or step cd) the mixture is incubated for 5-90 minutes to allow the formation of a ternary AAV-ADA-AAVp-captAb-BI complex.
Embodiment 37: The method according to any one of embodiments 2 to 3 and 5 to 36, wherein after the addition of the captAb-BI in step bc) or step cd) the mixture is incubated for 15-60 minutes to allow the formation of a ternary AAV-ADA-AAVp-captAb-BI complex.
Embodiment 38: The method according to any one of embodiments 2 to 3 and 5 to 37, wherein after the addition of the captAb-BI in step bc) or step cd) the mixture is incubated for about 30 minutes to allow the formation of a ternary AAV-ADA-AAVp-captAb-BI complex.
Embodiment 39: The method according to any one of embodiments 2 to 3 and 5 to 38, wherein the first member of a binding pair is biotin, streptavidin, digoxygenin, fluorescein or theophylline, respectively.
Embodiment 40: The method according to any one of embodiments 3 and 6 to 39, wherein the second member of the binding pair is immobilized on the solid surface.
Embodiment 41: The method according to any one of embodiments 3 and 6 to 40, wherein the second member of the binding pair is streptavidin in case the first member is biotin, biotin or an anti-biotin antibody in case the first member is streptavidin, an anti-digoxygenin antibody in case the first member is digoxygenin, an anti-fluorescein antibody in case the first member is fluorescein or an anti-theophylline antibody in case the first member is theophylline.
Embodiment 42: The method according to any one of embodiments 3 and 6 to 41, wherein the capturing in step bbc) or step ccd) is by incubating the mixture obtained in step bc) or step cd) with the solid surface for 1 to 60 minutes.
Embodiment 43: The method according to any one of embodiments 3 and 6 to 42, wherein the capturing in step bbc) or step ccd) is by incubating the mixture obtained in step bc) or step cd) with the solid surface for 5 to 45 minutes.
Embodiment 44: The method according to any one of embodiments 3 and 6 to 43, wherein the capturing in step bbc) or step ccd) is by incubating the mixture obtained in step bc) or step cd) with the solid surface for 10 to 25 minutes.
Embodiment 45: The method according to any one of embodiments 3 and 6 to 44, wherein the capturing in step bbc) or step ccd) is by incubating the mixture obtained in step bc) or step cd) with the solid surface for about 15 minutes.
Embodiment 46: The method according to any one of embodiments 1 to 45, wherein the detecting in step c) or step d) is by incubating with a detection antibody (dectAb) conjugated to a detectable label and either determining the presence of the detectable label or quantifying the amount of the detectable label.
Embodiment 47: The method according to any one of embodiments 1 to 46, wherein the mammalian serum sample is obtained from a mammal to which the AAV particle had been administered.

In addition to the various embodiments depicted and claimed herein, the disclosed subject matter is also directed to other embodiments having other combinations of the features disclosed and claimed herein. As such, the particular features presented herein, especially presented as individualized embodiments, can be combined with each other in other manners within the scope of the disclosed subject matter such that the disclosed subject matter includes any suitable combination of the embodiments and features disclosed herein. The description of specific embodiments of the disclosed subject matter has been presented for purposes of illustration and description. It is not intended to be exhaustive or to limit the disclosed subject matter to those embodiments disclosed.

### DESCRIPTION OF THE FIGURES

FIG. 1 Scheme of one preferred embodiment of the method according to the current invention.

FIG. 2 Scheme of an antigen-capture ELISA known from the art.

FIG. 3 Results of the method according to the invention obtained with eight commercially available cynomolgus sera; 1st bar: detection with polyclonal anti-human IgG antibody—no AAV2 particle addition; 2nd bar: detection with polyclonal anti-human IgG antibody—with AAV2 particle addition; 3rd bar: detection with monoclonal anti-cynomolgus IgG antibody—no AAV2 particle addition; 4th bar: detection with monoclonal anti-cynomolgus IgG antibody—with AAV2 particle addition.

FIG. 4 Results of the method according to the invention obtained with sixteen commercially available cynomolgus sera; left bar: +AAV2, right bar: without addition of AAV2.

FIG. 5 Results showing the absence of epitope masking by the capture antibody in a method according to the current invention.

FIG. 6 Exemplary determination of the optimum concentration of the AAV2 capsid variant 7m8 as AAVp in a method according to the current invention.

FIG. 7 Data showing that the signal obtained in the method according to the invention is correlating with the amount of AAVp in the sample in the pre-incubation step; 1st bar: amount of spiked AAV 2 (vp/mL) 132E8; 2nd bar: amount of spiked AAV 2 (vp/mL) 66E8 3rd bar: amount of spiked AAV 2 (vp/mL) 33E8; 4th bar: amount of spiked AAV 2 (vp/mL) 16.5E8; 5th bar: amount of spiked AAV 2 (vp/mL) 8.25E8; 6th bar: amount of spiked AAV 2 (vp/mL) 0E8.

FIG. 8 Effect of the pre-incubation time and serum content in the final sample; signal-to-noise ratio-average value for the 8 cynomolgus sera samples.

FIG. 9 Data showing the general applicability of the method according to the current invention: AAVp=AAV2 capsid variant 7m8.

FIG. 10 Data showing the general applicability of the method according to the current invention: AAVp=empty wild-type AAV2.

### DETAILED DESCRIPTION OF THE INVENTION

The current invention is based, at least in part, on the finding that by a pre-incubation of a sample, in which AAV-ADAs have to be determined, with an AAV particle of the same serotype as the therapeutic AAV particle complexes comprising the AAV-ADAs can be generated for thereafter following detection. Thereby the demand for therapeutic AAV particles as assay reagent compared to conventional AAV-ADA assays wherein labeled therapeutic AAV particle is employed can be dramatically reduced.

Unless otherwise defined herein, scientific and technical terms used in connection with the current invention shall have the meanings that are commonly understood by those of ordinary skill in the art. Further, unless otherwise required by context, singular terms shall include pluralities and plural terms shall include the singular.

Useful methods and techniques for carrying out the current invention are described in e.g. Ausubel, F. M. (ed.), Current Protocols in Molecular Biology, Volumes I to III (1997); Glover, N. D., and Hames, B. D., ed., DNA Cloning: A Practical Approach, Volumes I and II (1985), Oxford University Press; Freshney, R. I. (ed.), Animal Cell Culture—a practical approach, IRL Press Limited (1986); Watson, J. D., et al., Recombinant DNA, Second Edition, CHSL Press (1992); Winnacker, E. L., From Genes to Clones; N. Y., VCH Publishers (1987); Celis, J., ed., Cell Biology, Second Edition, Academic Press (1998); Freshney, R. I., Culture of Animal Cells: A Manual of Basic Technique, second edition, Alan R. Liss, Inc., N. Y. (1987), the content of which is incorporated herein by reference.

The use of recombinant DNA technology enables the generation of derivatives of a nucleic acid. Such derivatives can, for example, be modified in individual or several nucleotide positions by substitution, alteration, exchange, deletion or insertion. The modification or derivatization can, for example, be carried out by means of site directed mutagenesis. Such modifications can easily be carried out by a person skilled in the art (see e.g. Sambrook, J., et al., Molecular Cloning: A laboratory manual (1999) Cold Spring Harbor Laboratory Press, New York, USA; Hames, B. D., and Higgins, S. G., Nucleic acid hybridization—a practical approach (1985) IRL Press, Oxford, England).

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

The term “about” denotes a range of +/−20% of the thereafter following numerical value. In certain embodiments, the term “about” denotes a range of +/−10% of the thereafter following numerical value. In certain embodiments, the term “about” denotes a range of +/−5% of the thereafter following numerical value.

The term “comprising” also encompasses the term “consisting of”.

The term “antibody” is used herein in the broadest sense and encompasses various antibody structures, including but not limited to monoclonal antibodies, multispecific antibodies (e.g., bispecific antibodies), and antibody fragments so long as they exhibit the desired antigen-binding activity (AAV-binding activity).

A complete antibody in general comprises two so called light chain polypeptides (light chains) and two so called heavy chain polypeptides (heavy chains). Each of the heavy and light chain polypeptides contains a variable domain (generally the amino terminal portion of the polypeptide chain) and a constant region (generally the carboxyl terminal portion).

The constant region of the heavy chain mediates the binding of the antibody i) to cells bearing a Fc gamma receptor (FcγR), such as phagocytic cells, or ii) to cells bearing the neonatal Fc receptor (FcRn) also known as Brambell receptor. It also mediates the binding to some factors including factors of the classical complement system such as component (C1q). The constant domains of an antibody heavy chain comprise the CH1-domain, the CH2-domain and the CH3-domain, whereas the light chain comprises only one constant domain, CL, which can be of the kappa isotype or the lambda isotype.

The variable domains of an immunoglobulin's light or heavy chain mediate the binding of the antibody to its cognate antigen. Each variable domain comprises different segments, i.e. four framework regions (FR) and three hypervariable regions (HVR).

An “antibody fragment” refers to a molecule other than an intact or complete antibody that comprises a portion of an intact or complete antibody and that binds to the same antigen to which the intact or complete antibody binds. Examples of antibody fragments include but are not limited to Fv, Fab, Fab′, Fab′-SH, F(ab′) 2; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005), which is incorporated herein by reference.

The term “capture antibody” denotes an antibody that is used in a sandwich ELISA format to bind (i.e., capture) a target substance present in a sample for detection. A second antibody (i.e., the detection antibody) then binds to the captured target substance at a different epitope and allows detection of the capture antibody-target-complex (i.e. forming a “sandwich”).

A “conjugate” is a recombinant or chemical fusion comprising at least one proteinaceous part. The respective other part can be a second proteinaceous part or a non-proteinaceous part. As the parts of a conjugate a permanently linked to each other, it is a covalent conjugate. Covalent conjugation can be either direct or via a linker. In certain embodiments, direct recombinant conjugation is by construction of a polypeptide fusion (i.e. by genetic fusion of the two genes, e.g., encoding an antibody and a detectable label or member of a binding pair and expressed as a single polypeptide (chain)). In certain embodiments, direct chemical conjugation is by formation of a covalent bond between a reactive group on, e.g., the antibody and a corresponding group or acceptor on the detectable label or member of a binding pair. In certain embodiments, direct chemical conjugation is by modification (i.e. genetic modification) of one of the two parts to be conjugated to include a reactive group (as non-limiting examples, a sulfhydryl group or a carboxyl group) that forms a covalent attachment to the other part to be conjugated under appropriate conditions. As one non-limiting example, a molecule (i.e. an amino acid) with a desired reactive group (i.e. a cysteine residue) may be introduced into, e.g., the antibody.

Methods for covalent conjugation of nucleic acids to proteins are also known in the art (i.e., photo-crosslinking, see, e.g., Zatsepin et al. Russ. Chem. Rev. 74 (2005) 77-95).

In certain embodiments, the linker is a recombinant linker.

Conjugation via a recombinant linker can be performed using a single chain peptide linker. In certain embodiments, the single chain peptide linker comprises from one to twenty amino acid residues joined by peptide bonds. In certain embodiments, the amino acids are selected from the twenty naturally occurring amino acids. In certain other embodiments, one or more of the amino acids are selected from glycine, alanine, proline, asparagine, glutamine and lysine. In certain embodiments, the linker is a single chain peptide linker with an amino acid sequence with a length of at least 25 amino acid residues, in one preferred embodiment with a length of 32 to 50 amino acid residues. In certain embodiments the peptide linker is a (GxS) n linker with G=glycine, S=serine, (x=3, n=8, 9 or 10) or (x=4 and n=6, 7 or 8), in certain embodiments with x=4, n=6 or 7, in one preferred embodiment with x=4, n=7.

In certain embodiments, the linker is a chemical linker.

Chemical conjugation via a linker can be performed using a variety of chemical linkers. In certain embodiments, the antibody and the detectable label or the member of a binding pair are conjugated using a bifunctional protein coupling agents selected from the group consisting of N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HC1), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

The term “detection antibody” denotes an antibody, which carries a means for visualization or quantification. Such a means is typically an enzyme catalyzing the formation of a colored or fluorescent reaction product following the addition of a suitable substrate (detection reagent), such as, e.g., horseradish peroxidase, urease, alkaline phosphatase, glucoamylase or β-galactosidase. In certain embodiments, the detection antibody is a species-specific anti-immunoglobulin antibody. In certain embodiments, the detection antibody is conjugated to a detectable label. In certain embodiments, the detectable label is selected from the group consisting of biotin, digoxygenin, fluorescein, theophylline, a fluorescent marker, or a radioisotope. The detection antibody is detected and/or quantified using this detectable label.

The term “detection reagent” denotes a reagent, which permits the detection and/or quantification of an antibody, bound to an antigen. In certain embodiments, the detection reagent is a colorimetric substrate for an enzyme that has been conjugated to the antibody. Addition of a suitable substrate to the antibody-enzyme conjugate results in the production of a colorimetric or fluorimetric signal (e.g., following the binding of the conjugated antibody to the antigen of interest).

The term “ELISA” denotes an enzyme-linked immunosorbent assay. Different ELISA formats and applications are known in the art (see, e.g., Crowther, “Enzyme-Linked Immunosorbent Assay (ELISA),” in Molecular Biomethods Handbook, Rapley et al. [eds.], pp. 595-617, Humana Press, Inc., Totowa, NJ (1998); Harlow and Lane (eds.), Antibodies: A Laboratory Manual, Cold Spring Harbor Laboratory Press (1988); Ausubel et al. (eds.), Current Protocols in Molecular Biology, Ch. 11, John Wiley & Sons, Inc., New York (1994)). These are incorporated by reference herein.

One specific ELISA format is a so-called “direct ELISA”. In this ELISA format a target, e.g. an antibody, present in a sample is detected. In a direct ELISA the sample containing the target is brought in contact with a solid phase, such as e.g., stationary or immobilized support (e.g., a microtiter plate well). The target, if present in the sample, becomes immobilized to the solid phase, and is thereafter detected directly using an enzyme-conjugated detection molecule. If the target is an antigen the detection molecule is an antibody specific for the antigen, or if the target is an antibody specific for an antigen the detection molecule is an enzyme-conjugated antibody specific for the antigen.

Another specific ELISA format is a so-called “indirect ELISA”. In this ELISA format, an antigen (or an antibody) is immobilized to a solid phase (e.g., a microtiter plate well). Thereafter an antigen-specific antibody (or antigen) is added followed by the addition of a detection antibody specific for the antibody that specifically binds the antigen. This detection antibody can be a “species-specific” antibody (e.g., a goat anti-rabbit antibody).

Another specific ELISA format is a so-called “sandwich ELISA”. In this format the antigen is immobilized on a solid phase (e.g., a microtiter plate well) via capture by an antibody specifically binding to the antigen (i.e., a capture antibody), which is (covalently or via a specific binding pair) immobilized on the solid phase or a binding pair. Generally, a sample comprising the antigen is added to the solid phase, followed by washing. If the antigen of interest is present in the sample, it is bound by the capture antibody to the solid phase.

The term “Framework region” or “FR” refers to variable domain residues other than those of the hypervariable regions (HVRs). An antibody variable domain generally comprises four FRs: FR1, FR2, FR3, and FR4. Accordingly, the HVR and FR sequences generally appear in the following sequence in VH (or VL): FR1-HVR-H1 (HVR-L1)-FR2-HVR-H2 (HVR-L2)-FR3-HVR-H3 (HVR-L3)-FR4.

The terms “full length antibody”, “intact antibody”, and “whole antibody” are used herein interchangeably to refer to an antibody having a structure substantially similar to a native antibody structure.

The term “in-vitro” denotes either an artificial environment as such or that a process or reaction is performed within such an artificial environment.

The term “in-vivo” denotes the natural environment (e.g., an animal or a cell) of a compound or that a process or reaction is performed within its natural environment.

The term “immunoassay” denotes any technique that utilizes specifically binding molecules, such as antibodies, to capture and/or detect a specific target in a sample for qualitative or quantitative analysis. In general, an immunoassay is characterized by the following steps: 1) immobilization or capture of the analyte and 2) detection and measuring the analyte. The analyte can be captured, i.e. bound, on any solid surface, such as e.g. a membrane, plastic plate, or some other solid surface.

The term “linker” denotes a chemical linker or a single chain peptide linker that covalently connects different entities in a conjugate. The linker connects for example an antibody to the detectable label or to one partner of a binding pair.

The term “monoclonal antibody” as used herein refers to an antibody obtained from a population of substantially homogeneous antibodies, i.e., the individual antibodies comprising the population are identical, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each antibody molecule of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, monoclonal antibodies can be made by a variety of techniques, including but not limited to the hybridoma method, recombinant DNA methods, phage-display methods, and methods utilizing transgenic animals containing all or part of the human immunoglobulin loci.

The term “variable domain” refers to the part of an antibody heavy or light chain that is involved in binding of the antibody to its cognate antigen. The variable domains of the heavy chain and light chain (VH and VL, respectively) of a native antibody generally have similar structures, with each domain comprising four framework regions (FRs) and three hypervariable regions (HVRs). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively.

The term “hypervariable region” or “HVR” as used herein refers to each of the regions of an antibody variable domain which are hypervariable in sequence and which determine antigen binding specificity. These regions form the paratope or binding site of the binding region.

Generally, antibodies comprise six antigen binding specificity determining regions: three in the VH (H1, H2, H3), and three in the VL (L1, L2, L3). Exemplary antigen binding specificity determining regions herein include:
(a) hypervariable loops according to Chothia, which encompass amino acid residues 26-32 (L1), 50-52 (L2), 91-96 (L3), 26-32 (H1), 53-55 (H2), and 96-101 (H3) of the variable domains (Chothia and Lesk, J. Mol. Biol. 196:901-917 (1987));
(b) hypervariable regions according to Kabat, which encompass amino acid residues 24-34 (L1), 50-56 (L2), 89-97 (L3), 31-35b (H1), 50-65 (H2), and 95-102 (H3) of the variable domains (Kabat et al., Sequences of Proteins of Immunological Interest, 5th Ed. Public Health Service, National Institutes of Health, Bethesda, MD (1991));
(a+b) hypervariable loops combined with hypervariable regions, which encompass amino acid residues 24-34 (L1), 50-56 (L2), 89-97 (L3), 26-35 (H1), 50-65 (H2), and 95-102 (H3) of the variable domains (Chothia and Lesk, J. Mol. Biol. 196:901-917 (1987)+Kabat et al., Sequences of Proteins of Immunological Interest, 5th Ed. Public Health Service, National Institutes of Health, Bethesda, MD (1991));
and
(c) antigen contact residues, which encompass amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)).

Unless otherwise indicated, the HVRs are herein determined according to Kabat et al., supra. One of skill in the art will understand that the antigen binding specificity determining regions designations can also be determined according to Chothia, supra, McCallum, supra, or any other scientifically accepted nomenclature system.

The term “signal” as used herein encompasses any detectable physical change that can be used to indicate that a reaction has taken place, for example, binding of an antibody to its antigen. It is contemplated that signals in the form of fluorimetric or colorimetric products/reagents are specific forms of a signal and can be used in the method according to the current invention. In certain embodiments, the signal is assessed quantitatively.

The term “solid phase” denotes a non-fluid substance, and includes particles (including microparticles and beads) made from materials such as polymer, metal (paramagnetic, ferromagnetic particles), glass, and ceramic; gel substances such as silica, alumina, and polymer gels; capillaries, which may be made of polymer, metal, glass, and/or ceramic; zeolites and other porous substances; electrodes; microtiter plates; solid strips; and cuvettes, tubes or other spectrometer sample containers. A solid phase component is distinguished from inert solid surfaces in that a “solid phase” contains at least one moiety on its surface, which is intended to interact with a substance in a sample either directly or via a compound bound thereto. A solid phase may be a stationary component, such as a tube, strip, cuvette or microtiter plate, or may be non-stationary components, such as beads and microparticles. A variety of microparticles that allow either non-covalent or covalent attachment of proteins and other substances may be used. Such particles include polymer particles such as polystyrene and poly(methyl methacrylate); gold particles such as gold nanoparticles and gold colloids; and ceramic particles such as silica, glass, and metal oxide particles. See for example Martin, C. R., et al., Analytical Chemistry-News & Features, 70 (1998) 322A-327A, or Butler, J. E., Methods 22 (2000) 4-23, which is incorporated herein by reference.

The terms “therapeutic adeno-associated viral particle” and “drug” are used interchangeably herein. These terms are used in the broadest sense and encompass various structures so long as they exhibit the desired activity.

The term “sample” as used herein denotes any biological matrix from which the determination of the presence or amount of rAAV-ADA can be made. Exemplary, but not limiting samples are serum, plasma, aqueous humor, vitreous humor, retina tissue lysate, and tumor tissues. In one preferred embodiment, the sample is blood serum.

The term “anti-AAV antibody” as used herein denotes an antibody produced by the immune system of the recipient of a therapeutic AAV particle against said therapeutic AAV particle after administration thereof.

The term “antibody specifically binding to an AAV particle” as well as grammatical equivalents thereof as used herein denotes a recombinantly produced antibody that specifically binds to an AAV particle. In certain embodiments, the “antibody specifically binding to the AAV particles” has been generated by immunization of an experimental animal or by phage display methods.

The term “immunogenicity” as used herein denotes the potential of an AAV particle to induce an immune response in humans or animals. During drug development, immunogenicity is assessed primarily by the measurement of binding and neutralizing anti-AAV antibodies in a sample.

The term “empty particle” refers to an AAV particle that has an AAV protein shell but that lacks in whole or part a recombinant AAV vector, i.e. a nucleic acid that encodes a protein or is transcribed into a transcript of interest flanked by two AAV ITRs. Thus, an empty particle is an AAV particle without encapsidated functional or complete transgene (nucleic acid payload). Accordingly, the empty capsid does not function to transfer a nucleic acid that encodes a protein or is transcribed into a transcript of interest into the host cell.

A “recombinant AAV vector” is a nucleic acid that is derived from the wild-type genome of a virus, such as AAV, by using molecular biological methods, wherein the structural genes of the virus (e.g., of the AAV) are removed and replaced with a non-native nucleic acid, such as a nucleic acid transcribed into a therapeutic transcript or that encodes a therapeutic protein. Typically, for therapeutic recombinant AAV vectors both inverted terminal repeat (ITR) sequences of a wild-type AAV genome are retained.

A “recombinant” AAV vector is distinguished from a wild-type viral AAV genome in so far as that all or a part of the viral genome has been replaced with one or more non-native (i.e., heterologous) sequences. Incorporation of a non-native sequence therefore defines the viral vector as a “recombinant” vector, which in the case of AAV can be referred to as a “rAAV vector.”

A recombinant AAV vector (rAAV vector) can be packaged into a protein (capsid) shell-referred to herein as a “particle”—for subsequent infection (transduction) of a cell, ex vivo, in vitro or in vivo. Where a recombinant vector sequence is encapsulated or packaged into an AAV particle, the particle can also be referred to as a “rAAV particle”. The capsid of the particle includes proteins that encapsulate or package the rAAV vector. Particular examples of such proteins are viral envelope proteins, and in the case of AAV, capsid proteins, such as AAV VP1, VP2 and VP3.

As used herein, the term “serotype” is a distinction based on AAV capsid proteins being serologically distinct. Serologic distinctiveness is determined based on the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Under the traditional definition, a new serotype means that the virus of interest has been tested against serum specific for all existing and characterized serotypes for neutralizing activity and no antibodies have been found that neutralize the virus of interest. As more naturally occurring virus isolates are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or a variant of a given serotype.

The term “transgene” is used herein to conveniently refer to a nucleic acid that is intended, e.g. in a recombinant AAV vector, or has been introduced into a cell or organism. Transgenes include any nucleic acid, such as a gene that is transcribed into a transcript or that encodes a polypeptide or protein.

A “vector” refers to the portion of the sequence of a recombinant plasmid that is ultimately packaged or encapsulated, either directly or in form of a single strand or RNA, to form a viral (e.g., AAV) particle. In cases recombinant plasmids are used to construct or manufacture recombinant viral particles, the viral particle does not include the portion of the “plasmid” that does not correspond to the vector sequence of the recombinant plasmid. This non-vector portion of the recombinant plasmid is referred to as the “plasmid backbone”, which is important for cloning and amplification of the plasmid, a process that is needed for propagation and recombinant virus production, but is not itself packaged or encapsulated into virus (e.g., AAV) particles. Thus, a “vector” refers to the nucleic acid part of a plasmid that is packaged or encapsulated by a virus particle (e.g., AAV).

A “plasmid” is a form of nucleic acid or polynucleotide that typically has additional elements for expression (e.g., transcription, replication, etc.) or propagation (replication) of the plasmid. A plasmid as used herein also can be used to reference such nucleic acid or polynucleotide sequences.

### Recombinant Cells

Generally, for efficient as well as large-scale production of a proteinaceous compound of interest, such as e.g. a rAAV particle or a therapeutic polypeptide, a cell expressing and, if possible, also secreting said proteinaceous compound is required. Such a cell is termed “recombinant cell” or “recombinant production cell”.

For the generation of a “recombinant production cell” a suitable mammalian cell is transfected with the required nucleic acid sequences encoding said proteinaceous compound of interest. Depending on the polypeptide, e.g. an AAV, transfection of additional helper polypeptides may be necessary.

For the generation of stable recombinant production cells, a second step follows, wherein a single cell stably expressing the proteinaceous compound of interest is selected. This can be done, e.g., based on the co-expression of a selection marker, which had been co-transfected with the nucleic acid sequences encoding the proteinaceous compound of interest, or by the expression of the proteinaceous compound itself.

For expression of a coding sequence, i.e. of an open reading frame, by a recombinant cell additional regulatory elements, such as a promoter and polyadenylation signal (sequence), are necessary. Thus, an open reading frame is operably linked to said additional regulatory elements for transcription. This can be achieved by integrating it into a so-called expression cassette. The minimal regulatory elements required for an expression cassette to be functional in a mammalian cell are a promoter functional in said mammalian cell, which is located upstream, i.e. 5′, to the open reading frame, and a polyadenylation signal (sequence) functional in said mammalian cell, which is located downstream, i.e. 3′, to the open reading frame. Additionally a terminator sequence may be present 3′ to the polyadenylation signal (sequence). For expression, the promoter, the open reading frame/coding region and the polyadenylation signal sequence have to be arranged in an operably linked form.

In case the proteinaceous compound of interest is an AAV particle, which is composed of different (monomeric) capsid polypeptides and a single stranded DNA molecule and which in addition requires other adenoviral helper functions for production and encapsulation, a multitude of expression cassettes differing in the contained open reading frames/coding sequences are required. In this case, at least an expression cassette for each of the transgene, the different polypeptides forming the capsid of the recombinant AAV particle, the required helper functions as well as the VA RNA are required. Thus, individual expression cassettes for each of the helper E1A, E1B, E2A, E4orf6, the VA RNA as well as the rep and cap genes are required.

As outlined in the previous paragraphs, the more complex the proteinaceous compound of interest or the higher the number of additional required helper polypeptides and/or RNAs is, respectively, the higher is the number of required different expression cassettes. Inherently with the number of expression cassettes, also the total size of the required nucleic acids increases. However, there is a practical upper limit to the size of a nucleic acid that can be transferred, which is in the range of about 15 kbps (kilo-base-pairs). Above this limit handling and processing efficiency profoundly drops. This issue can be addressed by using two or more separate plasmids. Thereby the different expression cassettes are allocated to different plasmids, whereby each plasmid comprises only some of the expression cassettes.

For stable cell line development, random integration (RI) of the nucleic acid(s) carrying the expression cassettes for the proteinaceous compound of interest can be used. In general, by using RI the nucleic acids or fragments thereof integrate into the host cell's genome at random.

Alternatively, to RI, targeted integration (TI) can be used for CLD. In TI CLD, one or more nucleic acid(s) comprising the different expression cassettes is/are introduced/integrated at a predetermined locus in the host cell's genome.

In TI either homologous recombination or a recombinase mediated cassette exchange reaction (RMCE) can be employed for the integration of the nucleic acid(s) comprising the respective expression cassettes into the specific locus in the genome of the TI host cell.

### Adeno-Associated Viruses

For a general review of AAVs and of the adenovirus or herpes helper functions see, Berns and Bohensky, Advances in Virus Research, Academic Press., 32 (1987) 243-306. The genome of AAV is described in Srivastava et al., J. Virol., 45 (1983) 555-564. In U.S. Pat. No. 4,797,368 design considerations for constructing recombinant AAV vectors are described (see also WO 93/24641). Additional references describing AAV vectors are West et al., Virol. 160 (1987) 38-47; Kotin, Hum. Gene Ther. 5 (1994) 793-801; and Muzyczka J. Clin. Invest. 94 (1994) 1351. Construction of recombinant AAV vectors are described in U.S. Pat. No. 5,173,414; Lebkowski et al., Mol. Cell. Biol. 8 (1988) 3988-3996; Tratschin et al., Mol. Cell. Biol. 5 (1985) 3251-3260; Tratschin et al., Mol. Cell. Biol., 4 (1994) 2072-2081; Hermonat and Muzyczka Proc. Natl. Acad. Sci. USA 81 (1984) 6466-6470; Samulski et al. J. Virol. 63 (1989) 3822-3828. All of which are incorporated by reference herein.

An adeno-associated virus (AAV) is a replication-deficient parvovirus. It can replicate only in cells, in which certain viral functions are provided by a co-infecting helper virus, such as adenoviruses, herpesviruses and, in some cases, poxviruses such as vaccinia. Nevertheless, an AAV can replicate in virtually any cell line of human, simian or rodent origin once the appropriate helper viral functions are present.

Without helper viral genes being present, an AAV establishes latency in its host cell. Its genome integrates into a specific site in chromosome 19 [(Chr) 19 (q13.4)], which is termed the adeno-associated virus integration site 1 (AAVS1). For specific serotypes, such as AAV-2 other integration sites have been found, such as, e.g., on chromosome 5 [(Chr) 5 (p13.3)], termed AAVS2, and on chromosome 3 [(Chr) 3 (p24.3)], termed AAVS3.

AAVs are categorized into different serotypes. These have been allocated based on parameters, such as hemagglutination, tumorigenicity and DNA sequence homology. Up to now, more than 12 different serotypes and more than a hundred sequences corresponding to different clades of AAV have been identified.

The capsid protein type and symmetry determines the tissue tropism of the respective AAV. For example, AAV-2, AAV-4 and AAV-5 are specific to retina, AAV-2, AAV-5, AAV-8, AAV-9 and AAVrh-10 are specific for brain, AAV-1, AAV-2, AAV-6, AAV-8 and AAV-9 are specific for cardiac tissue, AAV-1, AAV-2, AAV-5, AAV-6, AAV-7, AAV-8, AAV-9 and AAV-10 are specific for liver, AAV-1, AAV-2, AAV-5 and AAV-9 are specific for lung.

Pseudotyping denotes a process comprising the cross packaging of the AAV genome or a recombinant AAV vector between various serotypes, i.e. the genome is packaged within capsid proteins originating from different serotypes. The serotype of the genome is determined by the serotype of its ITRs (inverted terminal repeats).

The wild-type AAV genome has a size of about 4.7 kb. The AAV genome further comprises two overlapping genes named rep and cap, which comprise multiple open reading frames (see, e.g., Srivastava et al., J. Viral., 45 (1983) 555-564; Hermonat et al., J. Viral. 51 (1984) 329-339; Tratschin et al., J. Virol., 51 (1984) 611-619). The Rep protein encoding open reading frame provides for four proteins of different size, which are termed Rep78, Rep68, Rep52 and Rep40. These are involved in replication, rescue and integration of the AAV. The Cap protein encoding open reading frame provides four proteins, which are termed VP1, VP2, VP3 and AAP. VP1, VP2 and VP3 are part of the proteinaceous capsid of the AAV particles. The combined rep and cap open reading frames are flanked at their 5′- and 3′-ends by so-called inverted terminal repeats (ITRs). For replication, an AAV requires in addition to the Rep and Cap proteins the products of the genes E1A, E1B, E4orf6, E2A and VA of an adenovirus or corresponding factors of another helper virus.

In the case of an AAV of the serotype 2 (AAV-2), for example, the ITRs each have a length of 145 nucleotides and flank a coding sequence region of about 4470 nucleotides. Of the ITR's 145 nucleotides 125 nucleotides have a palindromic sequence and can form a T-shaped hairpin structure. This structure has the function of a primer during viral replication. The remaining 20, non-paired, nucleotides are denoted as D-sequence.

The AAV genome harbors three transcription promoters P5, P19, and P40 (Laughlin et al., Proc. Natl. Acad. Sci. USA 76 (1979) 5567-5571) for the expression of the rep and cap genes.

The ITR sequences have to be present in cis to the coding region. The ITRs provide a functional origin of replication (ori), signals required for integration into the target cell's genome, and efficient excision and rescue from host cell chromosomes or recombinant plasmids. The ITRs further comprise origin of replication like-elements, such as a Rep-protein binding site (RBS) and a terminal resolution site (TRS). It has been found that the ITRs themselves can have the function of a transcription promoter in an AAV vector (Flotte et al., J. Biol. Chem. 268 (1993) 3781-3790; Flotte et al., Proc. Natl. Acad. Sci. USA 93 (1993) 10163-10167).

For replication and encapsidation, respectively, of the viral single-stranded DNA genome or a recombinant AAV vector the rep and cap genes have to be present in trans.

The rep gene locus comprises two internal promoters, termed P5 and P19. Promoter P5 is operably linked to a nucleic acid sequence providing for a non-spliced 4.2 kb mRNA encoding the Rep protein Rep78 (chromatin nickase to arrest cell cycle), and a spliced 3.9 kb mRNA encoding the Rep protein Rep68 (site-specific endonuclease). Promoter P19 is operably linked to a nucleic acid sequence providing for a non-spliced mRNA encoding the Rep protein Rep52 and a spliced 3.3 kb mRNA encoding the Rep protein Rep40 (DNA helicases for accumulation and packaging).

The two larger Rep proteins, Rep78 and Rep68, are essential for AAV duplex DNA replication, whereas the smaller Rep proteins, Rep52 and Rep40, seem to be essential for progeny, single-strand DNA accumulation (Chejanovsky & Carter, Virology 173 (1989) 120-128).

The larger Rep proteins, Rep68 and Rep78, can specifically bind to the hairpin conformation of the AAV ITR. They exhibit defined enzyme activities, which are required for resolving replication at the AAV termini. Expression of Rep78 or Rep68 could be sufficient for infectious particle formation (Holscher, C., et al. J. Virol. 68 (1994) 7169-7177 and 69 (1995) 6880-6885).

It is deemed that all Rep proteins, primarily Rep78 and Rep68, exhibit regulatory activities, such as induction and suppression of AAV genes as well as inhibitory effects on cell growth (Tratschin et al., Mol. Cell. Biol. 6 (1986) 2884-2894; Labow et al., Mol. Cell. Biol., 7 (1987) 1320-1325; Khleif et al., Virology, 181 (1991) 738-741).

Recombinant overexpression of Rep78 results in phenotype with reduced cell growth due to the induction of DNA damage. Thereby the host cell is arrested in the S phase, whereby latent infection by the virus is facilitated (Berthet, C., et al., Proc. Natl. Acad. Sci. USA 102 (2005) 13634-13639).

Tratschin et al. reported that the P5 promoter is negatively auto-regulated by Rep78 or Rep68 (Tratschin et al., Mol. Cell. Biol. 6 (1986) 2884-2894). Due to the toxic effects of expression of the Rep protein, only very low expression has been reported for certain cell lines after stable integration of AAV (see, e.g., Mendelson et al., Virol. 166 (1988) 154-165).

The cap gene locus comprises one promoter, termed P40. Promoter P40 is operably linked to a nucleic acid sequence providing for a 2.6 kb mRNA, which, by alternative splicing and use of alternative start codons, encodes the Cap proteins VP1 (87 kDa, non-spliced mRNA transcript), VP2 (72 kDa, the spliced mRNA transcript), and VP3 (61 kDa, from the alternative start codon). VP1 to VP3 constitute the building blocks of the viral capsid. The capsid has the function to bind to a cell surface receptor and allow for intracellular trafficking of the virus. VP3 accounts for about 90% of total viral particle protein. Nevertheless, all three proteins are essential for effective capsid production.

It has been reported that inactivation of all three capsid proteins VP1 to VP3 prevents accumulation of single-strand progeny AAV DNA. Mutations in the VP1 amino-terminus (“Lip-negative” or “Inf-negative”) still allows for assembly of single-stranded DNA into viral particles whereby the infectious titer is greatly reduced.

The AAP open reading frame is encoding the assembly activating protein (AAP). It has a size of about 22 kDa and transports the native VP proteins into the nucleolar region for capsid assembly. This open reading frame is located upstream of the VP3 protein encoding sequence.

In individual AAV particles, only one single-stranded DNA molecule is contained. This may be either a “plus” or a “minus” strand. AAV viral particles containing a DNA molecule are infectious. Inside the infected cell, the infecting single strand is converted into a double strand, which is subsequently amplified. The amplification results in a large pool of double stranded DNA molecules from which single strands are displaced and packaged into capsids.

Adeno-associated viral (AAV) vectors can transduce dividing cells as well as resting cells. It can be assumed that a transgene introduced using an AAV vector into a target cell will be expressed for a long period.

One drawback of using an AAV vector is the limitation of the size of the transgene that can be introduced into cells.

Viral vectors such as parvo-virus particles, including AAV serotypes and variants thereof, provide a means for delivery of nucleic acid into cells ex vivo, in vitro and in vivo, which encode proteins such that the cells express the encoded protein. AAVs are viruses useful as gene therapy vectors as they can penetrate cells and introduce nucleic acid/genetic material so that the nucleic acid/genetic material may be stably maintained in cells. In addition, these viruses can introduce nucleic acid/genetic material into specific sites. Because AAV are not associated with pathogenic disease in humans, AAV vectors are able to deliver endogenous and exogenous polynucleotide sequences (e.g., therapeutic proteins and agents) to human patients without causing substantial pathogenesis or disease.

Viral vectors, which may be used, include, but are not limited to, adeno-associated virus (AAV) particles of multiple serotypes (e.g., AAV-1 to AAV-12, and others) and hybrid/chimeric AAV particles.

AAV particles may be used as vehicles for effective gene delivery. Such particles possess a number of desirable features for such applications, including tropism for dividing and non-dividing cells. Early clinical experience with these vectors also demonstrated no sustained toxicity. AAV are known to infect a wide variety of cell types in vivo and in vitro by receptor-mediated endocytosis or by transcytosis. These vector systems have been tested in humans targeting retinal epithelium, liver, skeletal muscle, airways, brain, joints and hematopoietic stem cells.

Recombinant AAV particles do not typically include viral genes associated with pathogenesis. Such vectors typically have one or more of the wild-type AAV genes deleted in whole or in part, for example, rep and/or cap genes, but retain at least one functional flanking ITR sequence, as necessary for the rescue, replication, and packaging of the recombinant vector into an AAV particle. For example, only the essential parts of the vector e.g., the ITR and LTR elements, respectively, are included in a recombinant AAV vector. A recombinant AAV vector genome therefore includes sequences required for replication and packaging (e.g., functional ITR sequences) in cis.

Recombinant AAV vectors, as well as methods and uses thereof, include any viral strain or serotype. As a non-limiting example, a recombinant AAV vector can be based upon any AAV genome, such as AAV-1, -2, -3, -4, -5, -6, -7, -8, -9, -10, -11, -12, AAV-218, AAV rh74 or AAV 7m8 for example. Such vectors can be based on the same strain or serotype (or subgroup or variant), or be different from each other. As a non-limiting example, a recombinant AAV vector based upon one serotype genome can be identical to one or more of the capsid proteins that package the vector. In addition, a recombinant AAV vector genome can be based upon an AAV (e.g., AAV2) serotype genome distinct from one or more of the AAV capsid proteins that package the vector. For example, the AAV vector genome can be based upon AAV2, whereas at least one of the three capsid proteins could be an AAV1, AAV3, AAV4, AAV5, AAV6, AAV7, AAV8, AAV9, AAV10, AAV11, AAV12, AAV-218, AAV rh74, AAV 7m8 or a variant thereof, for example. AAV variants include variants and chimeras of AAV1, AAV2, AAV3, AAV4, AAV5, AAV6, AAV7, AAV8, AAV9, AAV10, AAV11, AAV12, AAV-218, AAV rh74 and AAV 7m8 capsids.

In certain embodiments of all aspects and embodiments, the adeno-associated virus (AAV) vectors or particles are selected from the group of serotypes comprising AAV1, AAV2, AAV3, AAV4, AAV5, AAV6, AAV7, AAV8, AAV9, AAV10, AAV11, AAV12, AAV-218, AAV rh74 and AAV 7m8, as well as variants (e.g., capsid variants, such as amino acid insertions, additions, substitutions and deletions) thereof. See, for example, WO 2013/158879, WO 2015/013313 and US 2013/0059732 (disclosing LK01, LK02, LK03, etc.).

AAV and AAV variant (e.g., capsid variant) serotypes (e.g., VP1, VP2, and/or VP3 sequences) may or may not be distinct from other AAV serotypes, including, for example, AAV1-AAV12 (e.g., distinct from VP1, VP2, and/or VP3 sequences of any of AAV1-AAV12 serotypes).

In certain embodiments of all aspects and embodiments, an AAV particle related to a reference serotype and has a polypeptide or subsequence thereof that includes or consists of a sequence that is at least 80% or more (e.g., 85%, 90%, 95%, 96%, 97%, 98%, 99%, 99.1%, 99.2%, 99.3%, 99.4%, 99.5%, etc.) identical to one or more AAV1, AAV2, AAV3, AAV4, AAV5, AAV6, AAV7, AAV8, AAV9, AAV10, AAV11, AAV12, AAV-218, AAV rh74 or AAV 7m8 (e.g., such as an ITR, or a VP1, VP2, and/or VP3 sequences).

Methods and uses of the invention include AAV sequences (polypeptides and nucleotides), and subsequences thereof that exhibit less than 100% sequence identity to a reference AAV serotype such as AAV1, AAV2, AAV3, AAV4, AAV5, AAV6, AAV7, AAV8, AAV9, AAV10, AAV11, AAV12, AAV-218, AAV rh74, or AAV 7m8, but are distinct from and not identical to known AAV genes or proteins, such as AAV1, AAV2, AAV3, AAV4, AAV5, AAV6, AAV7, AAV8, AAV9, AAV10, AAV11, AAV12, AAV-218, AAV rh74, or AAV 7m8, genes or proteins, etc. In certain embodiments of all aspects and embodiments, an AAV polypeptide or subsequence thereof includes or consists of a sequence at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to any reference AAV sequence or subsequence thereof, such as AAV1, AAV2, AAV3, AAV4, AAV5, AAV6, AAV7, AAV8, AAV9, AAV10, AAV11, AAV12, AAV-218, AAV rh74, or AAV 7m8 (e.g., VP1, VP2 and/or VP3 capsid or ITR). In certain embodiments, an AAV variant has 1, 2, 3, 4, 5, 5-10, 10-15, 15-20 or more amino acid substitutions compared to a reference serotype.

Recombinant AAV particles, including AAV1, AAV2, AAV3, AAV4, AAV5, AAV6, AAV7, AAV8, AAV9, AAV10, AAV11, AAV12, AAV-218, AAV rh74 and AAV 7m8, and variant, related, hybrid and chimeric sequences, can be constructed using recombinant techniques that are known to the skilled artisan, to include one or more nucleic acid sequences (recombinant AAV vector/transgene) flanked with one or more functional AAV ITR sequences.

Recombinant particles (e.g., rAAV particles) can be incorporated into pharmaceutical compositions. Such pharmaceutical compositions are useful for, among other things, administration and delivery to a subject in vivo or ex vivo. Pharmaceutical compositions contain in addition to the drug a pharmaceutically acceptable carrier or excipient. Such excipients include any pharmaceutical agent that does not itself induce an immune response harmful to the individual receiving the composition, and which may be administered without undue toxicity.

Protocols for the generation of adenoviral vectors have been described in U.S. Pat. Nos. 5,998,205; 6,228,646; 6,093,699; 6,100,242; WO 94/17810 and WO 94/23744.

### Recombinant AAV Particles

Different methods that are known in the art for generating rAAV particles. For example, transfection using AAV plasmid and AAV helper sequences in conjunction with co-infection with one AAV helper virus (e.g., adenovirus, herpesvirus, or vaccinia virus) or transfection with a recombinant AAV vector comprising plasmid, an AAV helper function comprising plasmid, and an rep/cap comprising plasmid. Non-limiting methods for generating rAAV particles are described, for example, in U.S. Pat. Nos. 6,001,650, 6,004,797, WO 2017/096039, and WO 2018/226887. Following recombinant rAAV particle production (i.e. particle generation in cell culture systems), rAAV particles can be obtained from the host cells and cell culture supernatant and purified.

For the generation of recombinant AAV particles, expression of the Rep and Cap proteins, the helper proteins E1A, E1B, E2A and E4orf6 as well as the adenoviral VA RNA in a single mammalian cell is required. The helper proteins E1A, E1B, E2A and E4orf6 can be expressed using any promoter as shown by Matsushita et al. (Gene Ther. 5 (1998) 938-945), especially the CMV IE promoter. Thus, any promoter can be used.

Generally, to produce recombinant AAV particles, different, complementing plasmids are co-transfected into a host cell. One of the plasmids comprises the transgene sandwiched between the two cis acting AAV ITRs. The missing AAV elements required for replication and subsequent packaging of progeny recombinant genomes, i.e. the open reading frames for the Rep and Cap proteins, are contained in trans on a second plasmid. The overexpression of the Rep proteins results in inhibitory effects on cell growth (Li, J., et al., J. Virol. 71 (1997) 5236-5243). Additionally, a third plasmid comprising the genes of a helper virus, i.e. E1, E4orf6, E2A and VA from adenovirus, is required for AAV replication.

To reduce the number of required plasmids, Rep, Cap and the adenovirus helper genes may be combined on a single plasmid.

Alternatively, the host cell may already stably express the E1 gene products. Such a cell is a HEK293 cell. The human embryonic kidney clone denoted as 293 was generated back in 1977 by integrating adenoviral DNA into human embryonic kidney cells (HEK cells) (Graham, F. L., et al., J. Gen. Virol. 36 (1977) 59-74). The HEK293 cell line comprises base pair 1 to 4344 of the adenovirus serotype 5 genome. This encompasses the E1A and E1B genes as well as the adenoviral packaging signals (Louis, N., et al., Virology 233 (1997) 423-429).

When using HEK293 cells the missing E2A, E4orf6 and VA genes can be introduced either by co-infection with an adenovirus or by co-transfection with an E2A-, E4orf6- and VA-expressing plasmid (see, e.g., Samulski, R. J., et al., J. Virol. 63 (1989) 3822-3828; Allen, J. M., et al., J. Virol. 71 (1997) 6816-6822; Tamayose, K., et al., Hum. Gene Ther. 7 (1996) 507-513; Flotte, T. R., et al., Gene Ther. 2 (1995) 29-37; Conway, J. E., et al., J. Virol. 71 (1997) 8780-8789; Chiorini, J. A., et al., Hum. Gene Ther. 6 (1995) 1531-1541; Ferrari, F. K., et al., J. Virol. 70 (1996) 3227-3234; Salvetti, A., et al., Hum. Gene Ther. 9 (1998) 695-706; Xiao, X., et al., J. Virol. 72 (1998) 2224-2232; Grimm, D., et al., Hum. Gene Ther. 9 (1998) 2745-2760; Zhang, X., et al., Hum. Gene Ther. 10 (1999) 2527-2537). Alternatively, adenovirus/AAV or herpes simplex virus/AAV hybrid vectors can be used (see, e.g., Conway, J. E., et al., J. Virol. 71 (1997) 8780-8789; Johnston, K. M., et al., Hum. Gene Ther. 8 (1997) 359-370; Thrasher, A. J., et al., Gene Ther. 2 (1995) 481-485; Fisher, J. K., et al., Hum. Gene Ther. 7 (1996) 2079-2087; Johnston, K. M., et al., Hum. Gene Ther. 8 (1997) 359-370).

Thus, cell lines in which the rep gene is integrated and expressed tend to grow slowly or express Rep proteins at very low levels.

In order to limit the transgene activity to specific tissues, i.e. to limit the site of integration the transgene can be operably linked to an inducible or tissue specific promoter (see, e.g., Yang, Y., et al. Hum. Gene. Ther. 6 (1995) 1203-1213).

In certain embodiments of all aspects and embodiments, the rAAV particles are derived from an AAV selected from the group consisting of AAV1, AAV2, AAV3, AAV4, AAV5, AAV6, AAV7, AAV8, AAV9, AAV10, AAV rh. 10, AAV rh.74 and AAV 7m8.

In certain embodiments of all aspects and embodiments, the rAAV particles comprise a capsid sequence having 70% or more sequence identity to an AAV1, AAV2, AAV3, AAV4, AAV5, AAV6, AAV7, AAV8, AAV9, AAV10, AAV rh. 10, AAV rh.74, or AAV 7m8 capsid sequence.

In certain embodiments of all aspects and embodiments, the rAAV particles comprise an ITR sequence having 70% or more sequence identity to an AAV1, AAV2, AAV3, AAV4, AAV5, AAV6, AAV7, AAV8, AAV9, or AAV10 ITR sequence.

### E1A, E1B, E2 and E4

The coding sequences of E1A and E1B (open reading frames) can be derived from a human adenovirus, such as, e.g., in particular of human adenovirus serotype 2 or serotype 5. An exemplary sequence of human Ad5 (adenovirus serotype 5) is found in GenBank entries X02996, AC_000008 and that of an exemplary human Ad2 in GenBank entry AC_000007. Nucleotides 505 to 3522 comprise the nucleic acid sequences encoding E1A and E1B of human adenovirus serotype 5. Plasmid pSTK146 as reported in EP 1 230 354, as well as plasmids pGS119 and pGS122 as reported in WO 2007/056994, can also be used a source for the E1A and E1B open reading frames.

E1A is the first viral helper gene that is expressed after adenoviral DNA enters the cell nucleus. The ELA gene encodes the 12S and 13S proteins, which are based on the same E1A mRNA by alternative splicing. Expression of the 12S and 13S proteins results in the activation of the other viral functions E1B, E2, E3 and E4. Additionally, expression of the 12S and 13S proteins force the cell into the S phase of the cell cycle. If only the E1A-derived proteins are expressed, the cell will dye (apoptosis).

E1B is the second viral helper gene that is expressed. It is activated by the E1A-derived proteins 12S and 13S. The E1B gene derived mRNA can be spliced in two different ways resulting in a first 55 kDa transcript and a second 19 kDa transcript. The E1B 55 kDa protein is involved in the modulation of the cell cycle, the prevention of the transport of cellular mRNA in the late phase of the infection, and the prevention of E1A-induced apoptosis. The E1B 19 kDa protein is involved in the prevention of ElA-induced apoptosis of cells.

The E2 gene encodes different proteins. The E2A transcript codes for the single strand-binding protein (SSBP), which is essential for AAV replication

In addition, the E4 gene encodes several proteins. The E4 gene derived 34 kDa protein (E4orf6) prevents the accumulation of cellular mRNAs in the cytoplasm together with the E1B 55 kDa protein, but also promotes the transport of viral RNAs from the cell nucleus into the cytoplasm.

### Adenoviral VA RNA Gene

The viral associated RNA (VA RNA) is a non-coding RNA of adenovirus (Ad), which is regulating translation. The adenoviral genome comprises two independent copies of such RNA: VAI (VA RNA copy I) and VAII (VA RNA copy II). Both are transcribed by RNA polymerase III (see, e.g., Machitani, M., et al., J. Contr. Rel. 154 (2011) 285-289) from a type 2 polymerases III promoter. For recombinant production, the adenoviral VA RNA gene can be driven by any promoter.

The structure, function, and evolution of adenovirus-associated RNA using a phylogenetic approach was investigated by Ma, Y. and Mathews, M. B. (J. Virol. 70 (1996) 5083-5099). They provided alignments as well as consensus VA RNA sequences based on 47 known human adenovirus serotypes.

The VA RNAs, VAI and VAII, comprise 157-160 nucleotides (nt).

Depending on the serotype, adenoviruses contain one or two VA RNA genes. VAI is believed to play the dominant pro-viral role, while VAII can only partially compensate for the absence of VAI (Vachon, V. K. and Conn, G. L., Virus Res. 212 (2016) 39-52).

The VARNAs are not essential, but play an important role in efficient viral growth by overcoming cellular antiviral machinery. That is, although VA RNAs are not essential for viral growth, VA RNA-deleted adenovirus cannot grow during the initial step of vector generation, where only a few copies of the viral genome are present per cell, possibly because viral genes other than VA RNAs that block the cellular antiviral machinery may not be sufficiently expressed (see Maekawa, A., et al. Nature Sci. Rep. 3 (2013) 1136).

Maekawa, A., et al. (Nature Sci. Rep. 3 (2013) 1136) reported efficient production of adenovirus vectors lacking genes of virus-associated RNAs that disturb cellular RNAi machinery, wherein HEK293 cells that constitutively and highly express flippase recombinase were infected to obtain VA RNA-deleted adenovirus by FLP recombinase-mediated excision of the VA RNA locus.

The human adenovirus 2 VA RNAI corresponds to nucleotides 10586-10810 of GenBank entry AC_000007 sequence. The human adenovirus 5 VA RNAI corresponds to nucleotides 10579-10820 of GenBank entry AC_000008 sequence.

Methods for Producing rAAV Particles

Carter et al. have shown that the entire rep and cap open reading frames in the wild-type AAV genome can be deleted and replaced with a transgene (Carter, B. J., in “Handbook of Parvoviruses”, ed. by P. Tijssen, CRC Press, pp. 155-168 (1990)). However, the ITRs have to be maintained to retain the function of replication, rescue, packaging, and integration of the transgene into the genome of the target cell.

When cells comprising the respective viral helper genes are transduced by an AAV vector, or, vice versa, when cells comprising an integrated AAV provirus are transduced by a suitable helper virus, then the AAV provirus is activated and enters a lytic infection cycle (Clark, K. R., et al., Hum. Gene Ther. 6 (1995) 1329-1341; Samulski, R. J., Curr. Opin. Genet. Dev. 3 (1993) 74-80).

More generally, a cell transfected or transduced with DNA for the recombinant production of AAV particles can be referred to as a “recombinant cell”. Such a cell can be, for example, a yeast cell, an insect cell, or a mammalian cell. It has been used as recipient of a nucleic acid (plasmid) encoding packaging proteins, such as AAV rep and cap, a nucleic acid (plasmid) encoding helper proteins, such as adenoviral E1A, E1B, E2A, E2B, E4orf6 and VA, and a nucleic acid (plasmid) that encodes a protein or is transcribed into a transcript of interest, i.e. a transgene, which is placed between two AAV ITRs. The term includes the progeny of the original cell, which has been transduced or transfected. It is understood that the progeny of a single parental cell may not necessarily be completely identical in morphology or in genomic or total nucleic acid complement as the original parent, due to natural, accidental, or deliberate mutation.

Numerous cell growth media appropriate for sustaining cell viability or providing cell growth and/or proliferation are commercially available. Examples of such media include serum free eukaryotic growth media, such as medium for sustaining viability or providing for the growth of mammalian (e.g., human) cells. Non-limiting examples include Ham's F12 or F12K medium (Sigma-Aldrich), FreeStyle (FS) F17 medium (Thermo-Fisher Scientific), MEM, DMEM, RPMI-1640 (Thermo-Fisher Scientific) and mixtures thereof. Such media can be supplemented with vitamins and/or trace minerals and/or salts and/or amino acids, such as essential amino acids for mammalian (e.g., human) cells.

Helper protein nucleic acids can be in the form of a plasmid, phage, transposon or cosmid. In particular, it has been demonstrated that the full-complement of adenovirus genes are not required for helper functions. For example, adenovirus mutants incapable of DNA replication and late gene synthesis have been shown to be permissive for AAV replication. See, e.g., Ito et al., J. Gen. Virol. 9 (1970) 243 and Ishibashi et al., Virology 45 (1971) 317.

Mutants within the E2B and E3 regions have been shown to support AAV replication, indicating that the E2B and E3 regions are probably not involved in providing helper function (see, Carter et al., Virology 126 (1983) 505). However, adenoviruses defective in the E1 region, or having a deleted E4 region, are unable to support AAV replication. Thus, for adenoviral helper proteins, E1A and E4 regions are required for AAV replication, either directly or indirectly (see, e.g., Laughlin et al., J. Virol. 41 (1982) 868; Janik et al., Proc. Natl. Acad. Sci. USA 78 (1981) 1925; Carter et al., Virology 126 (1983) 505). Other characterized adenoviral mutants include: E1B (Laughlin et al. (1982), supra; Janik et al. (1981), supra; Ostrove et al., Virology 104 (1980) 502); E2A (Handa et al., J. Gen. Virol. 29 (1975) 239; Strauss et al., J. Virol. 17 (1976) 140; Myers et al., J. Virol. 35 (1980) 665; Jay et al., Proc. Natl. Acad. Sci. USA 78 (1981) 2927; Myers et al., J. Biol. Chem. 256 (1981) 567); E2B (Carter, Adeno-Associated Virus Helper Functions, in I CRC Handbook of Parvoviruses (P. Tijssen ed., 1990)); E3 (Carter et al. (1983), supra); and E4 (Carter et al. (1983), supra; Carter (1995)).

Studies of the helper proteins provided by adenoviruses having mutations in the E1B have reported that the E1B 55 kDa protein is required for AAV particle production, while E1B 19 kDa is not. In addition, WO 97/17458 and Matshushita et al. (Gene Therapy 5 (1998) 938-945) described helper function plasmids encoding various adenoviral genes. An example of a helper plasmid comprises an adenoviral VA RNA coding region, an adenoviral E4orf6 coding region, an adenoviral E2A 72 kDa coding region, an adenoviral E1A coding region, and an adenoviral E1B region lacking an intact E1B 55 kDa coding region (see, e.g., WO 01/83797).

Diverse methods for the DNA transfer into mammalian cells have been reported in the art. These are all useful in the methods according to the current invention. For example, electroporation, nucleofection, or microinjection for nucleic acid transfer/transfection can be used. In an alternative, an inorganic substance (such as, e.g., calcium phosphate/DNA co-precipitation), a cationic polymer (such as, e.g., polyethylenimine, DEAE-dextran), or a cationic lipid (lipofection) can be used for nucleic acid transfer/transfection. Calcium phosphate and polyethylenimine are the most commonly used reagents for transfection of nucleic acids in larger scales (see, e.g., Baldi et al., Biotechnol. Lett. 29 (2007) 677-684), whereof polyethylenimine is preferred.

Expression control elements include constitutive or regulatable control elements, such as a tissue-specific expression control element or promoter.

Following production of recombinant viral (e.g., AAV) particles, if desired, the viral (e.g., rAAV) particles can be purified and/or isolated from host cells using a variety of conventional methods. Such methods include column chromatography, CsCl gradient centrifugation, iodixanol gradient centrifugation and the like.

For example, a plurality of column purification steps such as purification over an anion exchange column, an affinity column and/or a cation exchange column can be used (see, e.g., WO 02/12455 and US 2003/0207439). Alternatively, or in addition, an iodixanol or CsCl gradient can be used (see, e.g., US 2012/0135515; and US 2013/0072548).

An objective in the rAAV vector production and purification systems is to implement strategies to minimize/control the generation of production related impurities such as proteins, nucleic acids, and vector-related impurities, including wild-type/pseudo wild-type AAV species (wtAAV) and AAV-encapsulated residual DNA impurities.

Considering that the rAAV particle represents only a minor fraction of the biomass, rAAV particles need to be purified to a level of purity, which can be used as a clinical human gene therapy product (see, e.g., Smith P. H., et al., Mo. Therapy 7 (2003) 8348; Chadeuf G., et al, Mo. Therapy 12 (2005) 744; report from the CHMP gene therapy expert group meeting, European Medicines Agency EMEA/CHMP 2005, 183989/2004).

As an initial step, typically the cultivated cells that produced the rAAV particles are harvested, optionally in combination with harvesting cell culture supernatant (medium) in which the cells (suspension or adherent) producing rAAV particles have been cultured. The harvested cells and optionally cell culture supernatant may be used as is, as appropriate, or in addition lysed or/and concentrated. Further, if infection is employed to express helper functions, residual helper virus is inactivated. For example, adenovirus can be inactivated by heating to temperatures of approximately 60 °C for, e.g., 20 minutes or more, which inactivates only the helper virus since AAV is heat stable while the helper adenovirus is heat labile.

Cells alone or in the cultivation supernatant of the harvest are lysed by disrupting the cells, for example, by chemical or physical means, such as detergent, microfluidization and/or homogenization, to release the intracellular rAAV particles. Concurrently during cell lysis or subsequently after cell lysis, a nuclease, such as, e.g., benzonase, is added to degrade contaminating DNA. Typically, the resulting lysate is clarified to remove cell debris, e.g. by filtering or centrifuging, to render a clarified cell lysate. In a particular example, the lysate is filtered with a micron diameter pore size filter (such as a 0.1-10.0 μm pore size filter, for example, a 0.45 μm and/or pore size 0.2 μm filter), to produce a clarified lysate.

The lysate (optionally clarified) contains AAV particles (comprising rAAV particles with encapsidated nucleic acid as well as empty particles) and production/process related impurities, such as soluble cellular components from the host cells that can include, inter alia, cellular proteins, lipids, and/or nucleic acids, and cell culture medium components. The (optionally clarified) lysate is subjected to purification steps to purify AAV particles (comprising rAAV vectors) from impurities using chromatography. The clarified lysate may be diluted or concentrated with an appropriate buffer prior to the first chromatography step.

A first chromatography step may be a cation exchange chromatography or an anion exchange chromatography. If the first chromatography step is a cation exchange chromatography the second chromatography step can be an anion exchange chromatography or a size exclusion chromatography (SEC).

Alternatively, if the first chromatography step is a cation exchange chromatography the second chromatography step can be a size exclusion chromatography (SEC).

Still alternatively, a first chromatography step may be an affinity chromatography. If the first chromatography step is an affinity chromatography the second chromatography step can be an anion exchange chromatography.

Optionally, a third chromatography can be added to the foregoing chromatography steps. Typically, the optional third chromatography step follows cation exchange, anion exchange, size exclusion or affinity chromatography.

Cation exchange chromatography functions to separate the AAV particles from cellular and other components present in the clarified lysate and/or column eluate from an affinity or size exclusion chromatography. Examples of strong cation exchange resins capable of binding rAAV particles over a wide pH range include, without limitation, any sulfonic acid based resin as indicated by the presence of the sulfonate functional group, including aryl and alkyl substituted sulfonates, such as sulfopropyl or sulfoethyl resins. Representative matrices include but are not limited to POROS HS, POROS HS 50, POROS XS, POROS SP, and POROS S (strong cation exchangers available from Thermo Fisher Scientific, Inc., Waltham, MA, USA). Additional examples include Capto S, Capto S ImpAct, Capto S ImpRes (strong cation exchangers available from GE Healthcare, Marlborough, MA, USA), and commercial DOWEX®, AMBERLITE®, and AMBERLYSTR families of resins (available from Aldrich Chemical Company, Milliwaukee, WI, USA). Weak cation exchange resins include, without limitation, any carboxylic acid based resin. Exemplary cation exchange resins include carboxymethyl (CM), phospho (based on the phosphate functional group) and methyl sulfonate(S) resins.

Anion exchange chromatography functions to separate AAV particles from proteins, cellular and other components present in the clarified lysate and/or column eluate from an affinity or cation exchange or size exclusion chromatography. Anion exchange chromatography can also be used to reduce and thereby control the amount of empty particles in the eluate. For example, the anion exchange column having rAAV particle bound thereto can be washed with a solution comprising NaCl at a modest concentration (e.g., about 100-125 mM, such as 110-115 mM) and a portion of the empty particles can be eluted in the flow through without substantial elution of the rAAV particles. Subsequently, rAAV particles bound to the anion exchange column can be eluted using a solution comprising NaCl at a higher concentration (e.g., about 130-300 mM NaCl), thereby producing a column eluate with reduced or depleted amounts of empty capsids and proportionally increased amounts of rAAV particles comprising an rAAV vector.

Exemplary anion exchange resins include, without limitation, those based on polyamine resins and other resins. Examples of strong anion exchange resins include those based generally on the quaternized nitrogen atom including, without limitation, quaternary ammonium salt resins such as trialkylbenzyl ammonium resins. Suitable exchange chromatography materials include, without limitation, MACRO PREP Q (strong anion-exchanger available from BioRad, Hercules, CA, USA); UNO SPHERE Q (strong anion-exchanger available from BioRad, Hercules, CA, USA); POROS 50HQ (strong anion-exchanger available from Applied Biosystems, Foster City, CA, USA); POROS XQ (strong anion-exchanger available from Applied Biosystems, Foster City, CA, USA); POROS SOD (weak anion-exchanger available from Applied Biosystems, Foster City, CA, USA); POROS 50PI (weak anion-exchanger available from Applied Biosystems, Foster City, CA, USA); Capto Q, Capto XQ, Capto Q ImpRes, and SOURCE 30Q (strong anion-exchanger available from GE healthcare, Marlborough, MA, USA); DEAE SEPHAROSE (weak anion-exchanger available from Amersham Biosciences, Piscataway, NJ, USA); Q SEPHAROSE (strong anion-exchanger available from Amersham Biosciences, Piscataway, NJ, USA). Additional exemplary anion exchange resins include aminoethyl (AE), diethylaminoethyl (DEAE), diethylaminopropyl (DEPE) and quaternary amino ethyl (QAE).

Exemplary processes for recombinant AAV particle purification are reported in WO 2019/006390. Methods to determine infectious titer of rAAV particles containing a transgene are known in the art (see, e.g., Zhen et al., Hum. Gene Ther. 15 (2004) 709). Methods for assaying for empty particles and rAAV particles with packaged transgenes are known in the art (see, e.g., Grimm et al., Gene Therapy 6 (1999) 1322-1330; Sommer et al., Malec. Ther. 7 (2003) 122-128).

To determine the presence or amount of degraded/denatured capsid, purified rAAV particle can be subjected to SDS-polyacrylamide gel electrophoresis, consisting of any gel capable of separating the three capsid proteins, for example, a gradient gel, then running the gel until sample is separated, and blotting the gel onto nylon or nitrocellulose membranes. Anti-AAV capsid antibodies are then used as primary antibodies that bind to denatured capsid proteins (see, e.g., Wobus et al., J. Viral. 74 (2000) 9281-9293). A secondary antibody that binds to the primary antibody and that contains a means for detecting the primary antibody is used in a second incubation step. Binding between the primary and secondary antibodies is detected semi-quantitatively to determine the amount of capsids. Another method would be analytical HPLC with a SEC column or analytical ultracentrifuge.

### DESCRIPTION OF SPECIFIC EMBODIMENTS OF THE INVENTION

Herein is reported a method for measuring an anti-AAV particle antibody in a mammalian sample comprising the steps of diluting an aliquot of the sample, preferably to a final serum concentration between 5% (v/v) and 0.5% (v/v), adding AAV particles (AAVp) specifically bound by the anti-AAV particle antibody (rAAV-ADA) to the diluted sample to generate a spiked sample, pre-incubating the spiked sample to allow the formation of a rAAV-ADA-AAVp complex, and detecting the rAAV-ADA-AAVp complex, thereby measuring the anti-AAV antibody in the mammalian serum sample.

Typically, in AAV production rAAV particle titers around 1E11 to 1E12 in vg/ml and infectious titers around 1E8 to 1E9 TU/ml can be obtained.

Generally, the characterization of the immune response to an administered recombinant therapeutic AAV particle (rtAAV) generally requires the rtAAV itself as an assay reagent.

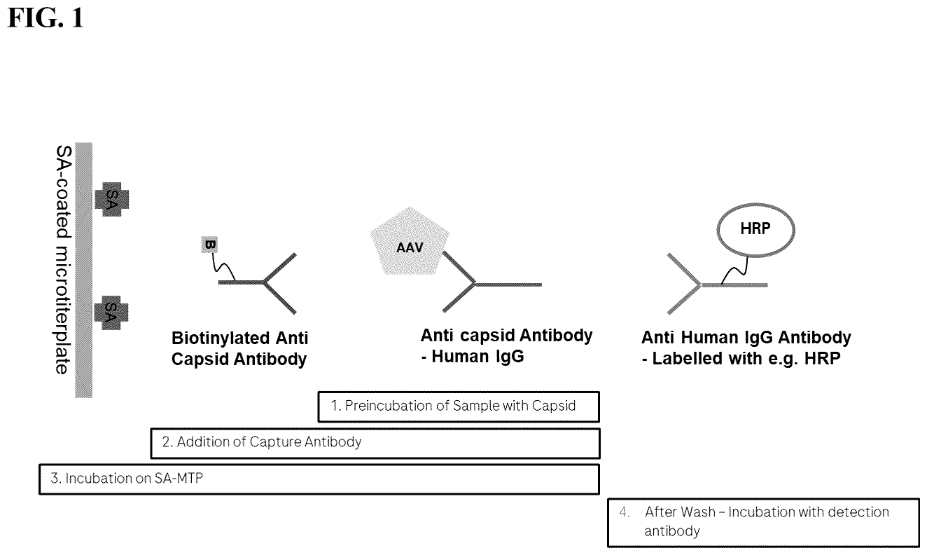

One embodiment of the principle of the method according to the current invention is depicted in FIG. 1. This is one preferred embodiment of the method according to the current invention.

In the first step of this embodiment of the method according to the current invention, the sample suspected to comprise ADAs against a recombinant therapeutic AAV particle (rtAAV-ADAs) is mixed with AAV particles (AAVp) of the same serotype as that of the rtAAV, preferably with the rtAAV itself. In this first incubation step a binary complex between rtAAV-ADAs present in the sample and the supplemented/spiked/added AAVp is formed (rtAAV-ADA-AAVp complex). As the complex formation is in solution, the required amount of AAV particles, e.g. of the rtAAV, is very small due to the fact that the required AAV particle concentration in solution can be very low. That is the required amount of AAV particles is dramatically reduced compared to a complex formation wherein the AAV particle, i.e. the rtAAV, has been immobilized on a solid surface as, e.g., in standard ELISAs in antigen-capture format. Furthermore, the required amount of the AAV particle can be further decreased by reducing the volume of the aliquot of the sample applied processed in the method according to the current invention.

In the second step of this embodiment of the method according to the current invention, an antibody specifically binding to the capsid of the AAVp, which is conjugated to a first member of a binding pair, (captAb-BI) is added to the pre-incubated sample. This captAb-BI specifically binds to the binary rtAAV-ADA-AAVp-complex. Thereby a ternary rtAAV-ADA-AAVp-captAb-BI-complex is formed.

It has to be pointed out that no loss in sensitivity is to be expected using the assay format according to the current invention compared to an assay using a chemically modified or immobilized AAV particle. In addition, due to the high number of epitopes present on a single AAV particle there is no risk of epitope saturation, masking or competition in a method according to the current invention. This additionally allows employing lower concentrations of the AAVp, i.e. even further reduced/lower amounts of the AAVp are required.

In the third step of this embodiment of the method according to the current invention, the ternary rtAAV-ADA-AAVp-captAb-BI-complex is immobilized or captured on a solid surface by the interaction between the first member of the binding pair of the captAb-BI and the respective cognate second member of the binding pair, which is conjugated to the solid surface.

In the fourth step of this embodiment of the method according to the current invention, the solid-phase captured ternary complex is detected by incubation with an antibody specifically binding to the Fc-region of the rtAAV-ADA, which in turn is conjugated to a detectable label (dectAb). The detectable label will only be retained on/immobilized on the solid surface in case a respective ternary complex has been captured thereon.

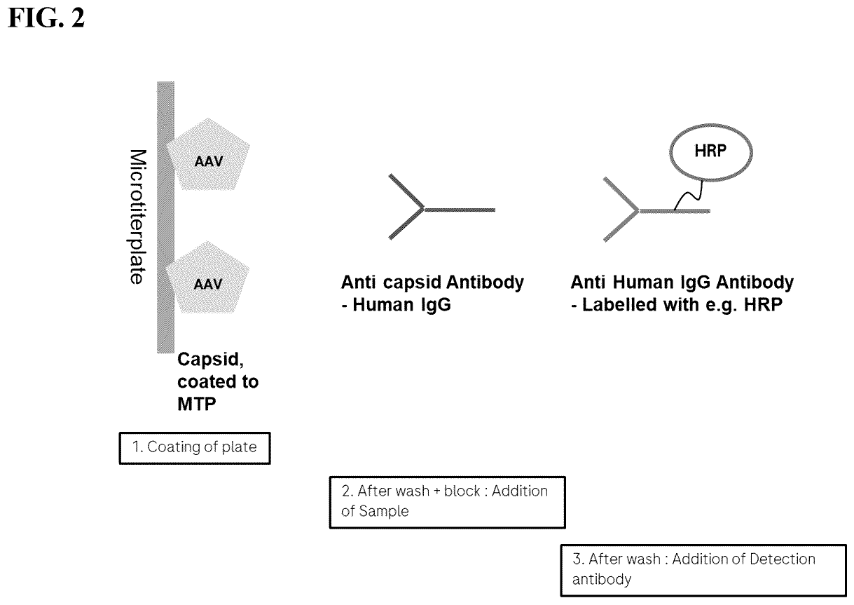

The principle of an antigen-capture assay known from the art is depicted in FIG. 2.

It can directly be seen that the most striking difference to the method according to the current invention is in the immobilization of the AAVp on the solid surface. As the total surface has to be covered by the AAVp, large amounts of AAVp are required, i.e. a high AAVp consumption is encompassed in methods according to the state of the art.

Additionally, in the state of the art assay formats a specific negative control is required and has to be provided, e.g. for assay performance check. Likewise, confirmation of the assay results is only possible by addition of an excess of AAVp or by AAV-ADA depletion.

In more detail, with the method according to the current invention
the required amount of particles for detecting and quantifying AAV-ADAs can be reduced by at least one order of magnitude, i.e. to 0.1-fold or 0.05-fold or even less of the amount required for an assay with solid-surface-immobilized AAVp;
a direct comparison of recombinant AAV materials and serotypes becomes possible, i.e. no requirement for derivatization or immobilization of the AAVp is needed; thereby the risk of changing the properties of the AAVp due to the chemical modification is prevented; a chemical modification might results in reduced sensitivity or selectivity of the assay;
a generic assay can be set up, in case an antibody that specifically binds to multiple or even all AAV serotypes is employed as captAb-BI, i.e. particles of any serotype can be captured and detected/quantified with the same assay reagents;
there is no need for AAV particle modification, i.e. no requirement to develop a specific chemical modification method;
a specificity control can be carried out by performing the assay according to the current invention without AAV particle vs. performing the assay according to the current invention with added AAV particles;
no additional particles are required, i.e. no further material has to be generated and provided;
current state of the art AAV-ADA detection assays using the antigen binding format (see FIG. 2) require a substantial amount of the AAV particle; such AAV particle may be from a different batch than the material used for in vivo administration and generation of the samples to be analyzed; theoretically, an immunogenic epitope of the administered AAV particle may not be contained/present in the AAV particle from the different batch being used in the assay; the methods according to the current invention precludes such a risk of underestimation of AAV-ADAs as the method according to the current invention requires only minute amounts of rAAV (for spiking), which can easily be obtained from the dosing material, i.e. is only a fraction of 0.001 or 0.000001 of a single dose (1E9 required for the assay, single dose in the range of 1E12 to 1E15).

The method according to the current invention is exemplified in the following with samples using an AAV particle of the AAV2 serotype. This is presented and provided solely to exemplify the method according to the invention and to show the function and general applicability of the method according to the current invention. This exemplification shall not be construed as a limitation of the scope of the invention. The true scope of the invention is set forth in the appended claims.

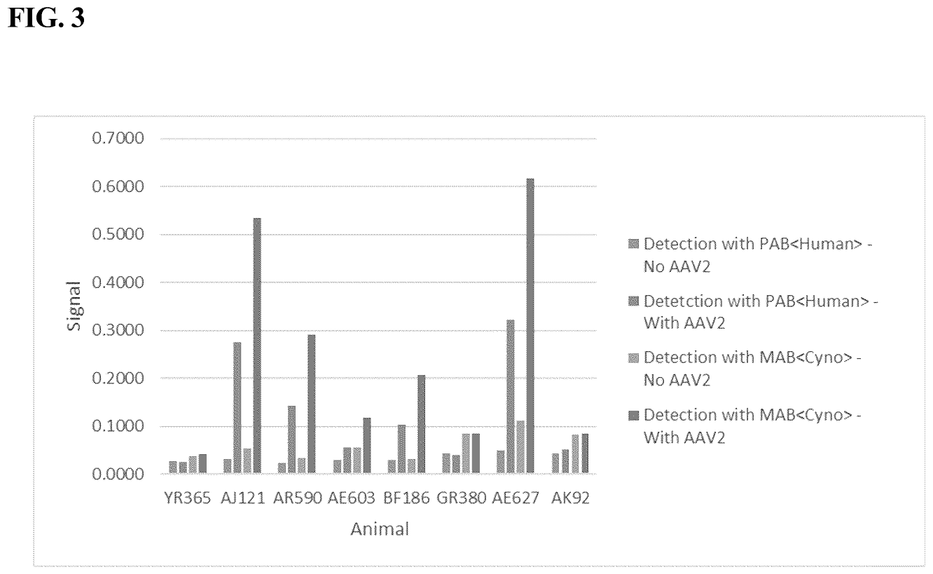

To exemplify the general applicability of the method according to the current invention, eight commercially available cynomolgus sera (Biotrend Chemikalien GmbH, Cologne, Germany) have been tested. The generic method is described in Example 1, the results are shown in FIG. 3 and Table 1.

TABLE 1

| | Detection with Goat Anti-Human IgG Antibody | | | |
|---|---|---|---|---|
| | With | Without | Ratio | |
| | AAV2 | AAV2 | with/without | |
| Serum | particle | particle | AAV2 particle | Positive (Pos)/ |
| sample | addition | addition | addition | Negative (Neg) |
| YR365 | 0.026 | 0.027 | 1.0 | Neg |
| AJ121 | 0.274 | 0.031 | 8.8 | Pos |
| AR590 | 0.143 | 0.023 | 6.2 | Pos |
| AE603 | 0.058 | 0.029 | 2.0 | Pos |
| BF186 | 0.104 | 0.029 | 3.6 | Pos |
| GR380 | 0.040 | 0.044 | 0.9 | Neg |
| AE627 | 0.323 | 0.050 | 6.5 | Pos |
| AK92 | 0.052 | 0.044 | 1.2 | Neg |

| | Detection with Mouse Anti-Cynomolgus IgG Antibody | | | |
|---|---|---|---|---|
| | With | Without | Ratio | |
| | AAV2 | AAV2 | with/without | |
| Serum | particle | particle | AAV2 particle | Positive (Pos)/ |
| sample | addition | addition | addition | Negative (Neg) |
| YR365 | 0.043 | 0.039 | 1.1 | Neg |
| AJ121 | 0.534 | 0.055 | 9.8 | Pos |
| AR590 | 0.292 | 0.034 | 8.7 | Pos |
| AE603 | 0.118 | 0.057 | 2.1 | Pos |
| BF186 | 0.207 | 0.031 | 6.7 | Pos |
| GR380 | 0.085 | 0.085 | 1.0 | Neg |
| AE627 | 0.617 | 0.113 | 5.5 | Pos |
| AK92 | 0.085 | 0.084 | 1.0 | Neg |

As AAVp a recombinant therapeutic AAV particle of the serotype 2 with a therapeutic transgene has been used at a concentration of 3.3E9 vp/mL. This corresponds to approximately 1E10 vp/plate (96 well plate). For comparison, an antigen-capture assay would require about 5E11 vp/plate (96 well plate).

As captAb-BI the commercially available murine anti-AAV2 antibody A20R from Progen has been used at a concentration of 0.1 μg/mL.

As sample eight commercially available cynomolgus sera at a dilution of 1:100 (v/v) have been used.

The incubation time on the plate was 5 minutes.

As dectAb either an IgG subclass independent anti-human IgG or anti-cynomolgus IgG antibody has been used at a final concentration of 0.4 μg/mL.

For color reaction 50 mM of an anti-digoxygenin antibody conjugated to HRP was used.

It can be seen that a positive signal is only obtained in case AAVp has been added to the cynomolgus serum.

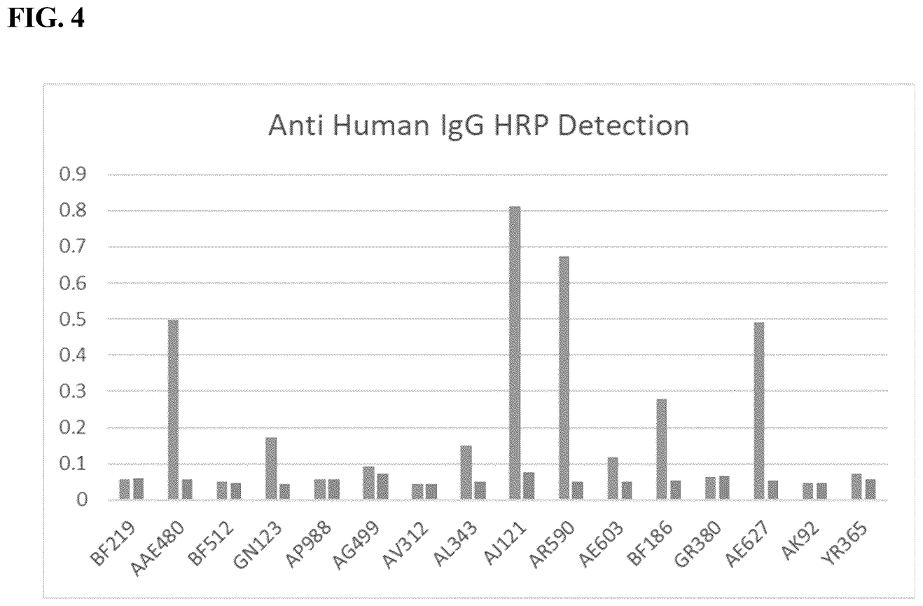

The applicability of the method according to the current invention has been tested with further commercially available cynomolgus sera using an anti-human IgG antibody as dectAb (Biotrend Chemikalien GmbH, Cologne, Germany). The results are shown in FIG. 4 and Table 2.

TABLE 2

| | Detection with Goat Anti-Human IgG antibody | | | |
|---|---|---|---|---|
| | With | Without | Ratio | |
| | AAV2 | AAV2 | with/without | |
| Serum | particle | particle | AAV2 particle | Positive (Pos)/ |
| sample | addition | addition | addition | Negative (Neg) |
| BF219 | 0.06 | 0.06 | 0.93 | Neg |
| AAE480 | 0.50 | 0.06 | 8.45 | Pos |
| BF512 | 0.05 | 0.05 | 1.01 | Neg |
| GN123 | 0.17 | 0.04 | 3.97 | Pos |
| AP988 | 0.06 | 0.06 | 1.00 | Neg |
| AG499 | 0.09 | 0.07 | 1.23 | Neg |
| AV312 | 0.04 | 0.05 | 0.99 | Neg |
| AL343 | 0.15 | 0.05 | 2.95 | Pos |
| AJ121 | 0.81 | 0.08 | 10.39 | Pos |
| AR590 | 0.67 | 0.05 | 12.81 | Pos |
| AE603 | 0.12 | 0.05 | 2.37 | Pos |
| BF186 | 0.28 | 0.06 | 5.02 | Pos |
| GR380 | 0.06 | 0.07 | 0.96 | Neg |
| AE627 | 0.49 | 0.06 | 8.95 | Pos |
| AK92 | 0.05 | 0.05 | 1.01 | Neg |
| YR365 | 0.07 | 0.06 | 1.25 | Neg |

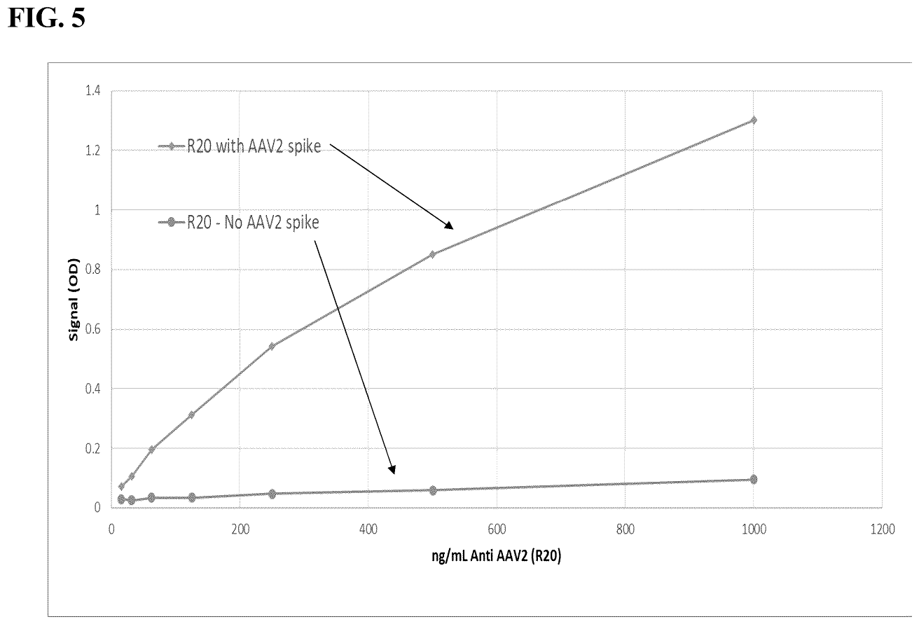

The absence of epitope masking by the capture antibody was shown likewise. In order to prove the absence of potential anti-AAV antibody epitope masking by the capture antibody, the worst-case scenario—the anti-AAV antibody of a study sample binds to the same epitope as the anti-AAV capture antibody—was simulated. To mimic this situation, the antibody R20A was used as the capture antibody as well as the positive control. More exactly, the mouse IgG of R20A was applied as the capture reagent and the human IgG of R20A as the positive control for human anti-AAV antibodies of a study sample. As the graph in FIG. 5 and the data in Table 3 show, the assay signal increases with increasing amount of the positive control. This means, there is actually no substantial competition for the binding sites: one or more capture antibodies and one or more positive control antibodies can bind simultaneously to a single AAV particle. Overall, there is no risk for epitope masking in the method according to the current invention.

TABLE 3

Standard = R20

| | With AAV2 particle | Without AAV2 particle |
|---|---|---|
| ng/ml | addition | addition |
| 1000 | 1.3515 | 0.0945 |
| 500 | 0.8790 | 0.0570 |
| 250 | 0.5435 | 0.0460 |
| 125 | 0.3335 | 0.0330 |
| 63 | 0.1985 | 0.0330 |
| 31 | 0.1085 | 0.0245 |
| 16 | 0.0700 | 0.0285 |
| 0 | 0.0210 | 0.0225 |

| | |
|---|---|
| Detection | Anti-Human IgG antibody-HRP conjugate |

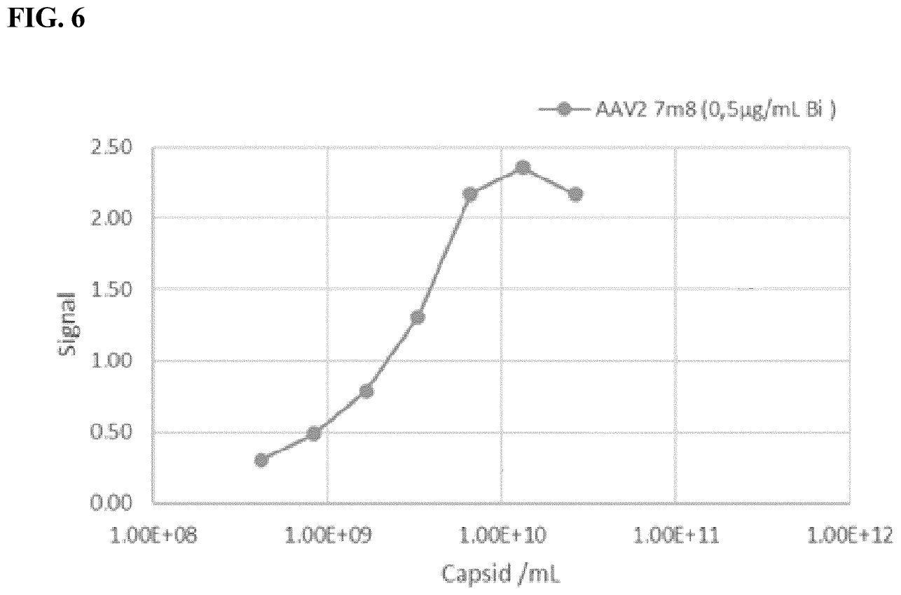

The optimum concentration of the AAV particle in the assay can easily be determined using a standard titration approach. This is shown for the AAV2 capsid variant denoted as 7m8 in FIG. 6. The optimum concentration has been determined to be 6.6E9 vp/mL in the assay for this capsid.

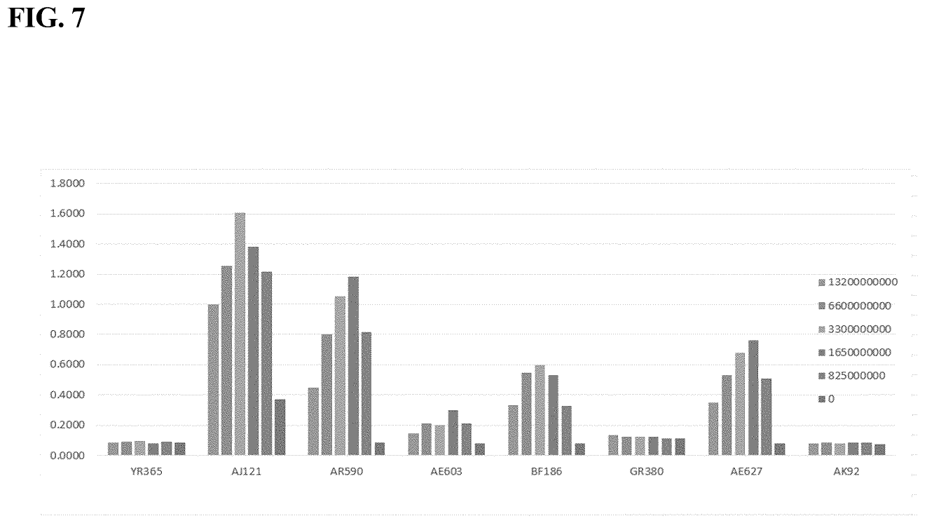

The signal obtained in the method according to the current invention is correlating with the amount of AAVp in the sample in the pre-incubation step. The same eight cynomolgus sera that have been used to generate the data presented in FIG. 3 and Table 1 have been used. Sera that were positive in the previous experiment (cf. FIG. 3 and Table 1) were also positive in this experiment. It can be seen that the optimum AAVp concentration is in the range from 1.65E9 to 3.3E9. The pre-incubation was performed for one hour. The results are shown in FIG. 7 and Table 4.

TABLE 4

| | Amount of spiked AAV 2 particles (vp/mL) | | | | | |
|---|---|---|---|---|---|---|
| Serum | 132E8 | 66E8 | 33E8 | 16.5E8 | 8.25E8 | 0E8 |
| YR365 | 0.0885 | 0.0915 | 0.0975 | 0.0905 | 0.0915 | 0.0860 |
| AJ121 | 1.0150 | 1.2910 | 1.6195 | 1.4295 | 1.2220 | 0.3845 |
| AR590 | 0.4615 | 0.7630 | 1.0570 | 1.1780 | 0.8320 | 0.0880 |
| AE603 | 0.1510 | 0.2055 | 0.1970 | 0.2960 | 0.2150 | 0.0790 |
| BF186 | 0.3385 | 0.5360 | 0.6010 | 0.5390 | 0.3455 | 0.0770 |
| GR380 | 0.1330 | 0.1285 | 0.1215 | 0.1245 | 0.1165 | 0.1155 |
| AE627 | 0.3400 | 0.5280 | 0.6795 | 0.7565 | 0.5240 | 0.0795 |
| AK92 | 0.0805 | 0.0850 | 0.0810 | 0.0840 | 0.0870 | 0.0740 |

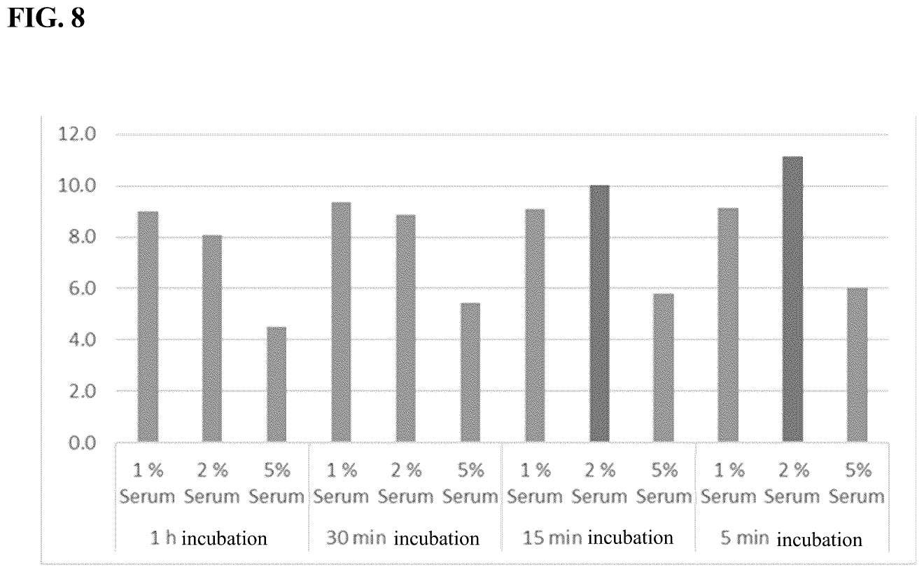

Further, the effect of the pre-incubation time and serum content in the final sample has been analyzed. The results are shown in FIG. 8 and Table 5 as the average of the signal-to-noise ratios obtained with the same eight cynomolgus sera as above. It can be seen that the highest signal was obtained for a pre-incubation time of 5 to 15 minutes with a final serum content of 2% in the pre-incubation mixture. For a pre-incubation time of 30 to 60 minutes the highest signal was obtained with a final serum concentration of 1% in the pre-incubation mixture.

TABLE 5

| | 60 min Incubation | | | 30 min Incubation | | |
|---|---|---|---|---|---|---|
| | 1% | 2% | 5% | 1% | 2% | 5% |
| Serum | Serum | Serum | Serum | Serum | Serum | Serum |
| YR365 | 0.1090 | 0.1670 | 0.3650 | 0.1060 | 0.1620 | 0.3370 |
| AJ121 | 1.7800 | 2.4040 | 2.2120 | 1.8320 | 2.4770 | 2.0330 |
| AR590 | 1.4210 | 1.7500 | 1.4510 | 1.5020 | 1.7460 | 1.4820 |
| AE603 | 0.3820 | 0.6040 | 1.3550 | 0.3970 | 0.6430 | 1.3850 |
| BF186 | 0.7610 | 1.3500 | 2.0480 | 0.7530 | 1.4380 | 1.9350 |
| GR380 | 0.1950 | 0.1960 | 0.3210 | 0.1690 | 0.1670 | 0.2720 |
| AE627 | 0.9800 | 1.3470 | 1.2820 | 0.9910 | 1.3990 | 0.8640 |
| AK92 | 0.1020 | 0.1450 | 0.3080 | 0.0850 | 0.1280 | 0.2570 |
| | 15 min Incubation | | | 5 min Incubation | | |
| | 1% | 2% | 5% | 1% | 2% | 5% |
| Serum | Serum | Serum | Serum | Serum | Serum | Serum |
| YR365 | 0.1050 | 0.1450 | 0.3180 | 0.0790 | 0.0920 | 0.2100 |
| AJ121 | 1.7120 | 2.3480 | 1.7020 | 1.1900 | 1.4530 | 0.9540 |
| AR590 | 1.4730 | 1.7960 | 1.3470 | 1.0380 | 1.2510 | 0.8940 |
| AE603 | 0.3850 | 0.6380 | 1.3190 | 0.2770 | 0.4710 | 0.8980 |
| BF186 | 0.7790 | 1.3020 | 1.9170 | 0.5400 | 0.9410 | 1.2910 |
| GR380 | 0.1500 | 0.1460 | 0.2330 | 0.1040 | 0.1000 | 0.1590 |
| AE627 | 0.9530 | 1.4520 | 1.3260 | 0.7200 | 1.0260 | 0.9540 |
| AK92 | 0.0770 | 0.1190 | 0.2300 | 0.0590 | 0.0860 | 0.1400 |

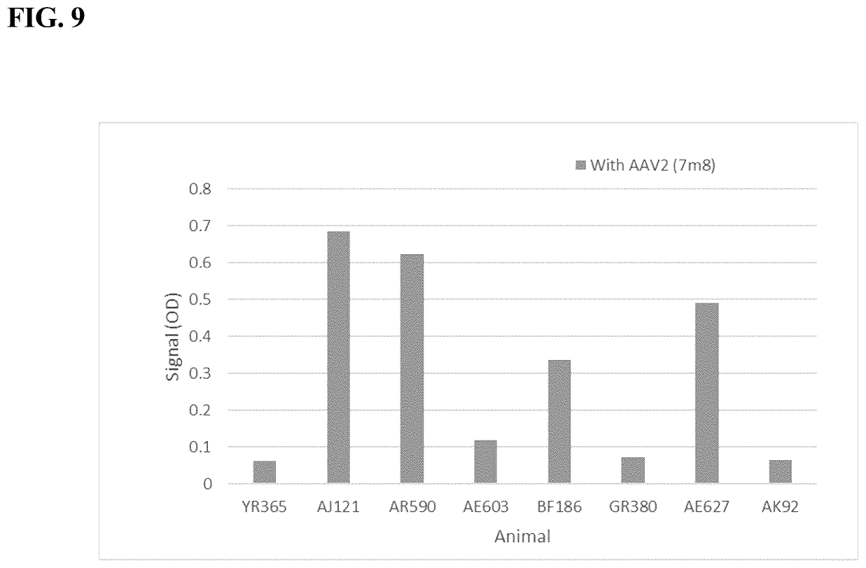

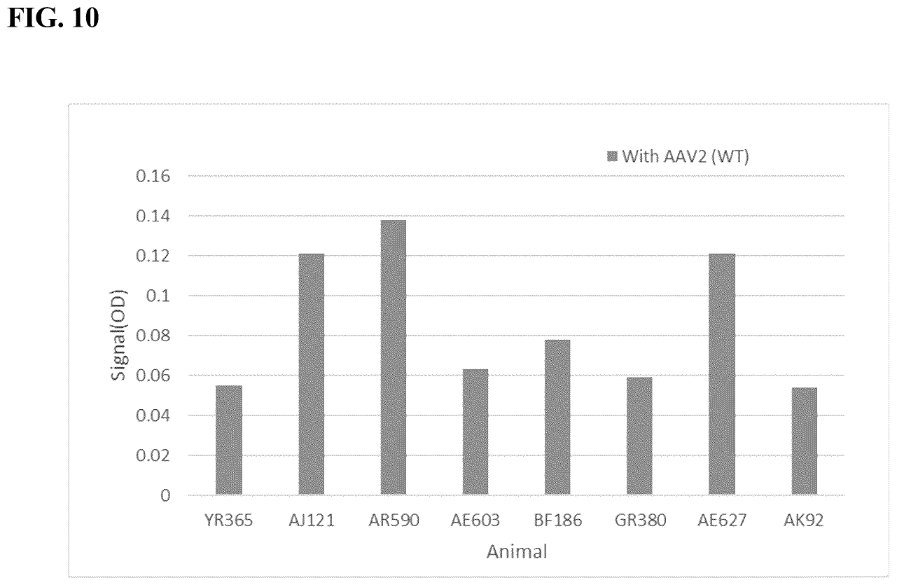

The general applicability of the method according to the current invention has also been shown. That is, comparable results as with the AAV-7m8 capsid variant have been obtained with a commercial empty wild-type AAV2 particle (Progene). This exemplifies the serotype/capsid independent applicability of the method according to the current invention. The respective data is shown in FIG. 9 (AAV2-7m8) and FIG. 10 (AAV2 wild-type empty particles).

### Calculation of Required Particle Amounts

In the method according to the current invention about 1.65E9 vp/well are required.

In an antigen-capture format according to the prior art about 5E10 vp/well are required.

Thus, with the method according to the current invention the required amount of AAVp can be reduced by more than one order of magnitude (7.9E9 vs. 4.8E11).

The respective data is shown in the following Table 6.

TABLE 6

| | Required | | | Required |
|---|---|---|---|---|
| | concentration | | Required | amount of virus |
| | [vp/mL] | Volume / | amount of virus | particles /plate |
| | in well | well [μL] | particles / well | ( 96 wells ) |
| In the | 1.65E9 | 50 | 8.25E7 | 7.92E9 |
| method | | | | |
| according | | | | |
| to the | | | | |
| current | | | | |
| invention | | | | |
| In an | 5.00E10 | 100 | 5.00E9 | 4.80E11 |
| antigen- | | | | |
| capture | | | | |
| format | | | | |
| according | | | | |
| to the | | | | |
| prior art | | | | |

The relation of 3.33E7 viral particles per nanogram of AAV mass has been used to convert mass concentration (ug/mL) into particle concentrations (vp/mL).

All references mentioned herein, even if not expressly stated, are incorporated herewith in the entirety by reference.

The following examples and figures are provided to aid the understanding of the present invention, the true scope of which is set forth in the appended claims. It is understood that modifications can be made in the procedures set forth without departing from the spirit of the invention.

### DESCRIPTION OF THE EXAMPLES

### Example 1

### Immune Complex Assay for Detection of Anti-AAV Antibodies in Mammalian Samples

Anti-AAV particle antibodies (AAV-ADAs) were determined in serum samples of human or monkey (cynomolgus) origin.

Prior to analysis, serum samples (1 μL) were diluted 1 to 100 (v/v) in LowCross-Buffer® (Candor Biosciences; Order No 100-500) and mixed with the respective AAV capsid (e.g. AAV2, Roche Diagnostics GmbH, Mannheim, Germany) to a concentration of 1.65 E9 capsids/mL. The total volume of the mixture was 120 μL. The mixture was incubated for one hour at room temperature. After incubation, a biotinylated serotype specific anti-capsid antibody (e.g., anti-AAV2 antibody (intact particle) mouse recombinant, A20R; Progen, Cat. No. 610298) was added to the mixture at a final concentration in the mixture of 0.25 μg/mL. The mixture of sample, AAV, and anti-capsid antibody was incubated for an additional 30 minutes at room temperature.

Thereafter, the mixture was added to a streptavidin-coated microtiter plate. The biotinylated capture antibody was captured by interaction with the streptavidin on the plate. The capturing was allowed to take place by incubation for 15 minutes. In the presence of AAV-ADAs, a complex of the biotinylated anti-capsid antibody, the added AAV capsid, and the AAV-ADA is immobilized on the plate during this step. After washing to remove unbound substances, the bound anti-capsid antibody-added AAV-AAV-ADA complex was labeled with a horseradish peroxidase-labeled anti-human or anti-monkey IgG polyclonal antibody. This labeling was by incubation for one hour at room temperature. After a washing step with PBS/Tween wash buffer, the horseradish peroxidase (HRP) substrate 2,2′-azinobis-(3-ethylbenzthiazoline-6-sulfonic acid) (ABTS) was added to the plate (50 μM final concentration). The reaction of the HRP with the ABTS generated a colored reaction product. The signal (extinction) was measured using an ELISA reader at 405 nm wavelength (reference wavelength: 490 nm ([405/490] nm)). The signal of each serum sample were determined in duplicate.

As a negative control, the assay is performed without the addition of the AAV capsid. No complexes can form and only the non-specific binding of the sample per se can take place. By comparing the two signals (with/without AAV addition), the signal can be corrected accordingly (signal-to-noise ratio).

1. A method for measuring an anti-AAV particle antibody in a mammalian serum sample comprising the following steps in the following order:
a) diluting at least an aliquot of the sample to a final mammalian serum concentration between and including 50% (v/v) and 0.5% (v/v),
b) adding AAV particles (AAVp) specifically bound by the anti-AAV particle antibody (AAV-ADA) to the diluted sample to generate a spiked sample,
c) pre-incubating the spiked sample to allow the formation of an AAV-ADA-AAVp complex,
and
d) detecting the AAV-ADA-AAVp complex,
thereby measuring the anti-AAV antibody in the mammalian serum sample.

2. The method according to claim 1, wherein the measuring is determining.

3. The method according to claim 1, wherein the measuring is quantifying.

4. The method according to any one of claims 1 to 3, wherein the pre-incubating is for 5 to 25 minutes and the final mammalian serum concentration is about 2% (v/v).

5. The method according to any one of claims 1 to 3, wherein the pre-incubating is for 26 to 90 minutes and the final mammalian serum concentration is about 1% (v/v).

6. The method according to any one of claims 1 to 5, wherein the final concentration of the AAVp in the spiked samples is in the range from 1.5 E9 to 3.5 E9 particles per mL spiked sample volume.

7. The method according to any one of claims 1 to 6, wherein the AAV-ADA specifically binds to an AAVp of the serotype 2 or 8 or 9 or rh74 or a variant thereof.

8. The method according to any one of claims 1 to 7, wherein the AAVp is conjugated to a first member of a binding pair.

9. The method according to any one of claims 1 to 8, wherein the method further comprises a step cd) after step c) and before step d) of
cd) adding a polypeptide or protein specifically binding to the AAVp that is conjugated to a first member of a binding pair (captAb-BI) to the pre-incubated spiked sample and incubating the mixture to allow the formation of an AAV-ADA-AAVp-captAb-BI complex,
and step d) is
d) detecting the AAV-ADA-AAVp-captAb-BI complex.

10. The method according to any one of claims 1 to 8, wherein the method further comprises a step dc) after step c) and before step d) of
dc) capturing the complex formed in step c) on a solid surface.

11. The method according to claim 9, wherein the method further comprises a step dc) after step cd) and before step d) of
dc) capturing the complex formed in step cd) on a solid surface.

12. The method according to any one of claims 10 to 11, wherein the second member of the binding pair is immobilized on the solid surface.

13. The method according to any one of claims 8 to 12, wherein the first member of a binding pair is selected from the group comprising biotin, streptavidin, digoxygenin, fluorescein or theophylline, and the second member of the binding pair is selected from the group comprising streptavidin, biotin, an anti-digoxygenin antibody, and anti-fluorescein antibody or an anti-theophylline antibody.

14. The method according to claim any one of claims 9 to 13, wherein the polypeptide or protein specifically binding to the AAVp is an anti-AAVp antibody or AAVp-binding fragment thereof.

15. The method according to any one of claims 1 to 14, wherein the detecting in step d) is by incubating with a detection antibody (dectAb) conjugated to a detectable label and either determining the presence of the detectable label or quantifying the amount of the detectable label.
